SEQUENCE LISTING (72 sequences)

SEQ ID NO: 1 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 364 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 386 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 386 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 359 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 376 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 364 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 386 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 386 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPLEATAPTAPQGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 359 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 376 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 376 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 376 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 389 aa, synthetic construct
TPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - protein, 389 aa, synthetic construct
TPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTSTQLLALKPCIGKACQNFSRCLEVQCQPDSSTLLPPRSPIALEATELPEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 22 - protein, 369 aa, synthetic construct
TQDCSFQYSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 369 aa, synthetic construct
TQDCSFQHSPISSDFAVKIRELSDYLLQDYPVTVASNLQDEELCGGLWRLVLAQRWMERLKTVAGSKMQGLLERVNTEIHFVTECAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 369 aa, synthetic construct
TQDCSFQYSPISSDFAVKIRELSDYLLQDYPVTVASNLQDEELCGGLWRLVLAQRWMERLKTVAGSKMQGLLERVNTEIHFVTECAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPLEATAESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDAAALPPPWSPRPLEATAESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 381 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDAAALPPPWAPRPLEATAEAKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 5 aa, synthetic construct
PTAPQ

SEQ ID NO: 29 - protein, 6 aa, synthetic construct
APTAPQ

SEQ ID NO: 30 - protein, 7 aa, synthetic construct
TAPTAPQ

SEQ ID NO: 31 - protein, 8 aa, synthetic construct
ATAPTAPQ

SEQ ID NO: 32 - protein, 9 aa, synthetic construct
EATAPTAPQ

SEQ ID NO: 33 - protein, 10 aa, synthetic construct
LEATAPTAPQ

SEQ ID NO: 34 - protein, 7 aa, synthetic construct
PTAPQPP

SEQ ID NO: 35 - protein, 8 aa, synthetic construct
APTAPQPP

SEQ ID NO: 36 - protein, 9 aa, synthetic construct
TAPTAPQPP

SEQ ID NO: 37 - protein, 10 aa, synthetic construct
ATAPTAPQPP

SEQ ID NO: 38 - protein, 11 aa, synthetic construct
EATAPTAPQPP

SEQ ID NO: 39 - protein, 12 aa, synthetic construct
LEATAPTAPQPP

SEQ ID NO: 40 - protein, 147 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRP

SEQ ID NO: 41 - protein, 148 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPL

SEQ ID NO: 42 - protein, 149 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLE

SEQ ID NO: 43 - protein, 150 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEA

SEQ ID NO: 44 - protein, 151 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEAT

SEQ ID NO: 45 - protein, 152 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATA

SEQ ID NO: 46 - protein, 153 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAP

SEQ ID NO: 47 - protein, 154 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPT

SEQ ID NO: 48 - protein, 155 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTA

SEQ ID NO: 49 - protein, 156 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAP

SEQ ID NO: 50 - protein, 157 aa, synthetic construct
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQ

SEQ ID NO: 51 - protein, 212 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 212 aa, synthetic construct
GGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 229 aa, synthetic construct
ESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 15 aa, synthetic construct
EPKSCDKTHTCPPCP

SEQ ID NO: 57 - protein, 20 aa, synthetic construct
EPKSCDKTHTCPPCPAPELL

SEQ ID NO: 58 - protein, 163 aa, synthetic construct
MTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQ

SEQ ID NO: 59 - protein, 156 aa, synthetic construct
MTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTA

SEQ ID NO: 60 - protein, 584 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 61 - protein, 609 aa, synthetic construct [sequence omitted]

SEQ ID NO: 62 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 63 - protein, 330 aa, synthetic construct [sequence omitted]

SEQ ID NO: 64 - protein, 4 aa, synthetic construct
GGGS

SEQ ID NO: 65 - protein, 5 aa, synthetic construct
GSGGS

SEQ ID NO: 66 - protein, 5 aa, synthetic construct
GGGGS

SEQ ID NO: 67 - protein, 4 aa, synthetic construct
GGSG

SEQ ID NO: 68 - protein, 5 aa, synthetic construct
GGSGG

SEQ ID NO: 69 - protein, 5 aa, synthetic construct
GSGSG

SEQ ID NO: 70 - protein, 5 aa, synthetic construct
GSGGG

SEQ ID NO: 71 - protein, 5 aa, synthetic construct
GGGSG

SEQ ID NO: 72 - protein, 5 aa, synthetic construct
GSSSG

(19) United States
(12) Patent Application Publication
(10) Pub. No.: US 2025/0340607 A1
(43) Pub. Date: Nov. 6, 2025

(54) ANTIGEN TOLERANCE INDUCTION THROUGH USE OF FLT3L VARIANTS

(71) Applicant: THE UNIVERSITY OF CHICAGO, Chicago, IL (US)

(72) Inventors: Jeffrey A. HUBBELL, Chicago, IL (US); Aaron ALPAR, Chicago, IL (US); Rachel P. WALLACE, Chicago, IL (US); Kirsten C. REFVIK, Chicago, IL (US)

(73) Assignee: THE UNIVERSITY OF CHICAGO, Chicago, IL (US)

(21) Appl. No.: 18/871,076

(22) PCT Filed: Jun. 2, 2023
(86) PCT No.: PCT/US2023/067897

§ 371 Date: Dec. 2, 2024

Related U.S. Application Data
(60) Provisional application No. 63/348,877, filed on Jun. 3, 2022.

(51) Int. Cl. C07K 14/475; A61K 38/18; A61K 45/06; A61K 47/64; A61K 47/68; A61P 37/04; C07K 14/765

(52) U.S. Cl. CPC C07K 14/475; A61K 38/18; A61K 45/06; A61K 47/643; A61K 47/68; A61P 37/04; C07K 14/765

(57) ABSTRACT

The current disclosure describes compositions and methods that may be administered to prevent immune responses against therapeutic molecules. The disclosure provides for a composition comprising a polypeptide comprising an engineered Fms Related Receptor Tyrosine Kinase 3 Ligand (Flt3L) protein. Also described is a method of treatment comprising: administering to a subject in need thereof, an effective amount of an engineered Flt3L protein of the disclosure. Methods also relate to a method for inducing immunotolerance in a subject in need thereof comprising, the method comprising administering to the subject an engineered Flt3L protein of the disclosure.

### CROSS-REFERENCE TO RELATED APPLICATIONS

This application claims the benefit of priority to U.S. Provisional Patent Application Ser. No. 63/348,877, filed Jun. 3, 2022, hereby incorporated by reference in its entirety.

### SEQUENCE LISTING

The application contains a Sequence Listing prepared in compliance with ST.26 format and is hereby incorporated by reference in its entirety. Said Sequence Listing, created on Jun. 1, 2023 is named “ARCDP0782WO” and is 20,480 bytes in size.

### BACKGROUND

### I. Field of the Invention

The invention generally relates to the field of medicine. More particularly, it concerns compositions and methods for inducing immunotolerance.

### II. Background

The human immune system has evolved to mount productive inflammatory responses against foreign pathogens through the specific recognition of unique pathogen antigens. However, there are circumstances in which such a specific recognition, when directed against the wrong antigens, can be detrimental to patients. Examples of these pathogenic immune responses include the discontinued use of necessary protein- or viral vector-based therapeutics, or the development of life-threatening allergic responses and debilitating autoimmune diseases. These unwanted immune responses have distinct downstream mechanisms, but all stem from an initial priming of the immune response. In this priming step, an antigen is up-taken by an antigen presenting cell (APC) such as a dendritic cell (DC) and presented to T cells with receptors specific to that antigen, along with secondary co-stimulatory signals via surface receptors and tertiary signals from secreted cytokines. These signals along with antigen presentation lead to T cell activation which can then initiate additional antigen-specific inflammatory cascades and the activation of antigen-specific antibody-generating B cell responses. There is a need in the art for therapies that block immune responses to therapeutic agents.

### SUMMARY OF INVENTION

The current disclosure describes compositions and methods that may be administered to prevent immune responses against therapeutic molecules. The disclosure provides for a composition comprising a polypeptide comprising an engineered Fms Related Receptor Tyrosine Kinase 3 Ligand (Flt3L) protein. Also described is a method of treatment comprising: administering to a subject in need thereof, an effective amount of an engineered Flt3L protein of the disclosure. Methods also relate to a method for inducing immunotolerance in a subject in need thereof comprising, the method comprising administering to the subject an engineered Flt3L protein of the disclosure.

The polypeptide may comprise or further comprise a serum protein. The serum protein may comprise an albumin protein. The engineered Flt3L protein may be connected to the albumin protein. The polypeptide may be a fusion of the engineered Flt3L protein and the albumin. The engineered Flt3L protein may be conjugated to the albumin protein. The polypeptide may exclude fusion with a serum and/or Fc polypeptide. The albumin protein may be a Mouse Serum Albumin (MSA) protein. The albumin protein may be a Human Serum Albumin (HSA) protein.

The fusion protein may comprise an amino acid sequence that is at least 90% identical to SEQ ID NOs: 58 and 60. The fusion protein may comprise an amino acid sequence that is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NOs: 58 and 60. The fusion protein may comprise an amino acid sequence that is at least 90% identical to SEQ ID NOs: 59 and 61. The fusion protein may comprise an amino acid sequence that is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NOs: 59 and 61. The Flt3L protein may be fused to a Fc domain of an IgG1 (Flt3L-Fc). The Fc protein may be a mouse Fc protein. The Flt3L-Fc fusion protein may comprise an amino acid sequence that is at least 90% identical to SEQ ID NOs: 58 and 62. The fusion protein may comprise an amino acid sequence that is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NOs: 58 and 62. The Fc protein may be a human Fc protein. The Flt3L-Fc fusion protein may comprise an amino acid sequence that is at least 90% identical to SEQ ID NOs: 59 and 63. The fusion protein may comprise an amino acid sequence that is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NOs: 59 and 63.

The Flt3L protein may be a mouse Flt3L protein. The Flt3L protein may be a human Flt3L protein. The compositions may comprise or further comprise an immunogenic biomolecule and/or immunogenic cell therapy. The methods may comprise or further comprise administration of an immunogenic biomolecule and/or immunogenic cell therapy. The subject may be one that has been administered, will be administer, or is prescribed an immunogenic biomolecule, an immunogenic cell therapy, or an exogenous antigen wherein the exogenous antigen comprises a therapeutic biomolecule and/or cell therapy. The immunogenic biomolecule may be a nucleic acid, protein, or virus, or a combination thereof. The nucleic acid is DNA, RNA, or a combination thereof. The nucleic acid may be an siRNA, miRNA, gRNA, mRNA, lincRNA, cDNA, gene or gene fragment, expression construct, or plasmid, or a combination thereof. The virus may be adenovirus, adeno-associated virus, lentivirus, or retrovirus, or a combination thereof. The protein may be an enzyme, an antigen binding protein, an antibody or antibody fragment, a cytokine, a chemokine, a ligand, a receptor, or binding protein, or a combination thereof. The cell therapy may comprise T-cells, B-cells, dendritic cells, NK or iNK cells, other hematopoietic cells, epithelial cells, neuronal or nerve cells, stem cells, pluripotent cells, cardiac cells, skeletal cells, smooth muscle cells, skin cells, endothelial cells, fat cells, pancreatic cells, or bone cells, or a combination thereof. The composition may comprise, consist, or consist essentially of one or more of the Flt3L protein, Flt3L fusion protein, an immunogenic biomolecule, and an immunogenic cell therapy. The composition may consist of the Flt3L protein and/or Flt3L fusion protein.

The compositions and/or methods described herein may comprise, consist essentially of, or consist of Flt3L protein, human Flt3L (hFlt3L) protein, mouse Flt3L (mFlt3L) protein, hFlt3L-HSA fusion protein, mFlt3L-MSA fusion protein, hFlt3L-Fc fusion protein, and/or mFlt3L-Fc fusion protein. The compositions and/or methods described herein may exclude rapamycin and/or treatment with rapamycin.

The compositions and/or methods described herein may be utilized in a tolerogenic format to prevent development of immunity in a subject in response to/against an exogenous biologic (e.g., antigen), such as but not limited to, proteins, cytokines, chemokines, enzymes, antibodies, antigen-binding fragments, effector immune cell therapy (e.g., immune effector cells of any kind, including conventional T cells, gamma-delta T cells, NK cells, NK T cells, invariant NK T cells, regulatory T cells, macrophages, B cells, dendritic cells, tumor-infiltrating lymphocytes, MSCs, or a mixture thereof; The cells may be allogeneic, autologous, or xenogeneic with respect to an individual, including an individual in need of the cells, such as an individual with cancer, with or without transgenic components such as chimeric antigen receptors, T cell receptors, etc.), organ transfusion, blood transfusion, and/or stem cell transfusion.

Flt3L may be utilized in its native state, and/or expressed as a fusion protein with albumin (or the Fc domain of an IgG1), in a manner to promote tolerance. Tolerance may be measured as the prevention of anti-drug antibody formation and prevention of immune cell response when used in conjunction with antigen delivery. Tolerance may be measured as the prevention of anti-drug antibody formation and prevention of T cell response when used in conjunction with antigen delivery. Tolerance may be measured as the prevention of anti-drug antibody formation and prevention of T cell response when used in conjunction with oral antigen delivery.

A tolerogenic inducing compound (e.g., a compound comprising Flt3L as described herein) and/or an exogenous biologic (e.g., antigen) are comprised in a pharmaceutical composition. A tolerogenic inducing compound and an exogenous biologic may be comprised in the same pharmaceutical composition. A tolerogenic inducing compound an exogenous biologic may be comprised in different pharmaceutical compositions. Pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

As utilized herein, Mouse Serum Albumin (MSA) is an albumin derived from a coding sequence found in the mouse genome, it is not necessarily derived from the serum, blood, and/or plasma of a mouse. A mouse albumin protein may be a recombinant protein produced ex-vivo by a cell line. An albumin protein may comprise a sequence represented and/or encoded by NCBI reference sequences: NC_000071.7 Reference GRCm39 C57BL/6J (range 90608729 to 90624461) genomic sequence, NM_009654.4 mRNA sequence, and/or NP_033784.2 protein sequence or a fragment thereof. The albumin protein may be 80%, 81%, 82%, 83%, 84%, 85%, 86%, 87%, 88%, 89%, 90%, 91%, 92%, 93%, 94%, 95%, 96%, 97%, 98%, 99%, or 100% or any range derivable therein, identical to SEQ ID NO:60.

As utilized herein, Human Serum Albumin (HSA) is an albumin derived from a coding sequence found in the human genome, it is not necessarily derived from the serum, blood, and/or plasma of a human. A human albumin protein may be a recombinant protein produced ex-vivo by a cell line. An albumin protein may comprise a sequence represented and/or encoded by NCBI reference sequences: NC_000004.12 Reference GRCh38.p14 Primary Assembly (range 73404287 to 73421482) genomic sequence, NM_000477.7 mRNA sequence, and/or NP_000468.1 protein sequence, or a fragment thereof. The albumin protein may be 80%, 81%, 82%, 83%, 84%, 85%, 86%, 87%, 88%, 89%, 90%, 91%, 92%, 93%, 94%, 95%, 96%, 97%, 98%, 99%, or 100% or any range derivable therein, identical to SEQ ID NO: 61.

An albumin protein may comprise a sequence derived from a sequence that comprises a coding sequence found in the genome of an animal. An albumin protein may comprise a sequence derived from a sequence that comprises a coding sequence found in the genome of a mammal. An albumin protein may comprise a sequence derived from a sequence that comprises a coding sequence found in, but not limited to, the genome of dogs, cats, ferrets, cattle, rabbits, ducks, pigs, goats, deer, turkeys, doves, sheep, fishes, chickens, horses, geese, llamas, ostriches, camels, oxen, and/or reindeer.

A Flt3L protein may comprise a sequence represented and/or encoded by NCBI reference sequences: NC_000073.7 Reference GRCm39 C57BL/6J (range 44780607 to 44785914 complement) genomic sequence, mRNA sequences: NM_001402831.1, NM_001402832.1, NM_001402833.1, NM_001402834.1, NM_001402835.1, NM_001402836.1, NM_001402837.1, NM_013520.4, protein sequences: NP_001389760.1, NP_001389761.1, NP_001389762.1, NP_001389763.1, NP_001389764.1, NP_001389765.1, NP_001389766.1, and/or NP_038548.3, or fragments thereof. A Flt3L protein may be 80%, 81%, 82%, 83%, 84%, 85%, 86%, 87%, 88%, 89%, 90%, 91%, 92%, 93%, 94%, 95%, 96%, 97%, 98%, 99%, or 100% or any range derivable therein, identical to SEQ ID NO:58.

A Flt3L protein may comprise a sequence represented and/or encoded by NCBI reference sequences: NC_000019.10 Reference GRCh38.p14 Primary Assembly (range 49474215 to 49486231) genomic sequence, mRNA sequences: NM_001204502.2, NM_001204503.2, NM_001278637.2, NM_001278638.2, NM_001459.4, protein sequences: NP_001191431.1, NP_001191432.1, NP_001265566.1, NP_001265567.1, and/or NP_001450.2, or fragments thereof. A Flt3L protein may be 80%, 81%, 82%, 83%, 84%, 85%, 86%, 87%, 88%, 89%, 90%, 91%, 92%, 93%, 94%, 95%, 96%, 97%, 98%, 99%, or 100% or any range derivable therein, identical to SEQ ID NO:59.

A Flt3L protein may comprise a sequence derived from a sequence that comprises a coding sequence found in the genome of an animal. A Flt3L protein may comprise a sequence derived from a sequence that comprises a coding sequence found in the genome of a mammal. A Flt3L protein may comprise a sequence derived from a sequence that comprises a coding sequence found in, but not limited to, the genome of dogs, cats, ferrets, cattle, rabbits, ducks, pigs, goats, deer, turkeys, doves, sheep, fishes, chickens, horses, geese, llamas, ostriches, camels, oxen, and/or reindeer.

A Flt3L protein may be fused to an Fc domain. An Fc domain may be derived from a human gene or human protein. An Fc domain may be derived from a mouse gene or protein. An Fc domain protein may be 80%, 81%, 82%, 83%, 84%, 85%, 86%, 87%, 88%, 89%, 90%, 91%, 92%, 93%, 94%, 95%, 96%, 97%, 98%, 99%, or 100% or any range derivable therein, identical to SEQ ID NO:62 or SEQ ID NO:63.

A Flt3L protein may be fused to another protein, such as a serum protein, albumin, and/or Fc polypeptide through a linker peptide sequence. Any suitable linker known in the art can be utilized. In some embodiments, a linker sequence is a Glycine Serine linker. Multiple linker sequences may be utilized. The linker may comprise a glycine serine linker. The linker may comprise or consist of GGGS-SEQ ID NO:64, GSGGS-SEQ ID NO:65, GGGGS-SEQ ID NO:66, GGSG-SEQ ID NO:67, GGSGG-SEQ ID NO:68, GSGSG-SEQ ID NO:69, GSGGG-SEQ ID NO:70, GGGSG-SEQ ID NO:71, GSSSG-SEQ ID NO:72, and the like. Glycine-alanine polymers, alanine-serine polymers, and other flexible linkers known in the art and may be used as a linker in the polypeptides of the disclosure. Also included are linkers comprising or consisting of 1, 2, 3, 4, 5, 6, 7, 8, 9, or 10 repeated units of any one of SEQ ID NOS: 64-72.

The composition and polypeptides of the disclosure may be administered orally, nasally, mucosally, intravenously, and/or subcutaneously. Other forms of administration are described herein and may be implemented in the methods of the disclosure. The methods may exclude administration orally, nasally, mucosally, intravenously, subcutaneously, or a route of administration described herein. The exogenous antigen, therapeutic biomolecule, cell therapy, immunogenic biomolecule, and/or immunogenic cell therapy may be administered prior to the engineered Flt3L protein, after the engineered Flt3L protein, or concurrently with the engineered Flt3L protein. The exogenous antigen, therapeutic biomolecule, cell therapy, immunogenic biomolecule, and/or immunogenic cell therapy may be administered at a time period of within 24 hours of the engineered Flt3L protein and either before or after the engineered Flt3L protein. The exogenous antigen and engineered Flt3L protein may be provided to the subject within a 72 hour period. The exogenous antigen, therapeutic biomolecule, cell therapy, immunogenic biomolecule, and/or immunogenic cell therapy may be administered, administered at least, or administered at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 hours and/or 1, 2, 3, 4, 5, 6, 7, 8, 9, 10, 11, 12, 13, 14, 15, 16, 17, 18, 19, or 20 days, and/or 1, 2, 3, 4, 5, 6, 7, and/or 8 weeks (or any derivable range therein) before the engineered Flt3L protein

The treatment may be one that prevents and/or inhibits induction of an immune response (e.g., promotes tolerogenesis or immunotolerance) in the subject against the exogenous antigentherapeutic biomolecule and/or cell therapy. The subject may be one that has an increased and/or enhanced tolerance to the exogenous antigen without reduction, inhibition, and/or blunting of other productive immune responses. Excessive inflammation (e.g., life threatening and/or capable of creating permanent physiological damage) in a subject in response to the exogenous antigen therapeutic biomolecule and/or cell therapy may be avoided, according to the methods of the disclosure. The exogenous antigen and/or engineered Flt3L protein may be provided orally, nasally, mucosally, intravenously, and/or subcutaneously. Providing of the Flt3L protein may prevent and/or treat an autoimmune condition in the subject. The subject may be further defined as a mammal. The subject may be a human subject. The engineered Flt3L protein may be provided at the same time or within 1 day of administration of the exogenous antigen.

The subject may be one that has received or will receive (has been prescribed) an additional therapy. The subject may be one that has not received or has not been prescribed an additional therapy. The additional therapy may comprise an immunotherapy and/or an immune agonist.

The FLT3L polypeptide may comprise a fusion protein comprising: a FLT3L polypeptide linked to a serum protein. The serum protein may be albumin. The serum protein may be human serum albumin. The serum protein may be mouse serum albumin. The serum protein may comprise the amino acid sequence of SEQ ID NO:60 or 61, or an amino acid sequence having at least 80% sequence identity to SEQ ID NO:60 or 61. The serum protein may have an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:60 or 61. The FLT3L polypeptide may comprise the amino acid sequence of one of SEQ ID NOs: 40-50, 58 or 59 or an amino acid sequence having at least 80% sequence identity to one of SEQ ID NOs: 40-50, 58 or 59. The FLT3L polypeptide may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 40-50, 58 or 59. The FLT3L polypeptide may be further defined as an FLT3L extracellular domain. The FLT3L polypeptide may comprise a fusion protein comprising: a FLT3L extracellular domain operably linked to an immunoglobulin fragment crystallizable region (Fc region).

At least 5 amino acids may be truncated from the C-terminus of the FLT3L extracellular domain; and/or the Fc region does not comprise a hinge region. The FLT3L extracellular domain may be a human FLT3L extracellular domain or derived from a human FLT3L extracellular domain. The fusion protein may be capable of binding to human FLT3. The FLT3L extracellular domain may be from FLT3L isoform 1. The FLT3L extracellular domain may be from FLT3L isoform 2. The FLT3L extracellular domain may exclude the amino acid sequence PTAPQ. At least 6, 7, 8, 9, 10, 11, 12, 13, 14 or 15 amino acids (or any derivable range therein) are truncated from the C-terminus of the FLT3L extracellular domain. The FLT3L extracellular domain may exclude the amino acid sequence APTAPQ (SEQ ID NO: 29), TAPTAPQ (SEQ ID NO:30), ATAPTAPQ (SEQ ID NO:31), EATAPTAPQ (SEQ ID NO: 32), or LEATAPTAPQ (SEQ ID NO:33). The FLT3L extracellular domain may exclude the amino acid sequence PTAPQPP (SEQ ID NO:34), APTAPQPP (SEQ ID NO:35), TAPTAPQPP (SEQ ID NO:36), ATAPTAPQPP (SEQ ID NO:37), EATAPTAPQPP (SEQ ID NO: 38), or LEATAPTAPQPP (SEQ ID NO:39). The FLT3L extracellular domain may comprise an N-terminal signal peptide. The FLT3L extracellular domain may comprise an amino acid substitution at one or more of the following amino acid positions: H8Y, K84E, N100, S102, N123 and S125, wherein the amino acid residue positions are with reference to SEQ ID NOs: 1-18, 21-27 or 40-50. The FLT3L extracellular domain may comprise one or more of the following amino acid substitutions: H8Y, K84E, S102A, and/or S125A; wherein the amino acid residue positions are with reference to SEQ ID NOs: 1-18, 21-27 or 40-50. One ore both of serine residues at positions 102 and 125 may be substituted to alanine, wherein the amino acid residue positions are with reference to SEQ ID NOs: 1-18, 21-27 or 40-50.

The Fc region may be from a human IgG1, IgG2, IgG3 or IgG4. The Fc region may comprise a human IgG1 isotype and comprises one or more amino acid substitutions in the Fc region at a residue position selected from the group consisting of: N297A, N297G, N297Q, N297G, D265A, L234A, L235A, C226S, C229S, P238S, E233P, L234V, P238A, A327Q, A327G, P329A, P329G, K322A, L234F, L235E, P331S, T394D, A330L, M252Y, S254T, T256E, M428L, N434S, T366W, T366S, L368A, Y407V, and any combination thereof, wherein the numbering of the residues is according to EU numbering. The Fc region may comprise a human IgG1 isotype and comprises one or more amino acid substitutions in the Fc region at a residue position selected from the group consisting of: L234A, L234V, L234F, L235A, L235E, P331S, and any combination thereof, wherein the numbering of the residues is according to EU numbering. The Fc region may comprise a human IgG4 isotype and comprises one or more amino acid substitutions in the Fc region at a residue position selected from the group consisting of: E233P, F234V, F234A, L235A, G237A, E318A, S228P, L235E, T394D, M252Y, S254T, T256E, N297A, N297G, N297Q, T366W, T366S, L368A, Y407V, M428L, N434S, and any combination thereof, wherein the numbering of the residues is according to EU numbering. The Fc region may comprise a human IgG4 isotype and comprises one or more amino acid substitutions in the Fc region at a residue position selected from the group consisting of: F234V, F234A, L235A, L235E, S228P, and any combination thereof, wherein the numbering of the residues is according to EU numbering. The Fc region may comprise the following amino acids at the indicated positions (EU index numbering): Tyrosine at position 252, threonine at position 254 and glutamic acid at position 256 (YTE); or Leucine at position 428 and serine at position 434 (LS). The FLT3L extracellular domain may comprise the amino acid sequence of one of SEQ ID NOs: 40-50, or an amino acid sequence having at least 80% sequence identity to one of SEQ ID NOs: 40-50. The FLT3L extracellular domain may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 40-50.

The Fc region may comprise the amino acid sequence of one of SEQ ID NOs: 51-55, 62, and 63, or an amino acid sequence having at least 80% sequence identity to one of SEQ ID NOs: 51-55, 62, and 63. The Fc region may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 51-55, 62, and 63. The fusion protein may comprise the amino acid sequence of one of SEQ ID NOs: 1-27, or an amino acid sequence having at least 80% sequence identity to one of SEQ ID NOs: 1-27. The fusion protein may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 1-27. The Fc region may be from a human IgG1 and does not comprise a hinge region. The C-terminus of the FLT3L extracellular domain may be un-truncated. The Fc region may be derived from a human IgG1 isotype and does not comprise a hinge region, e.g., does not the amino acid sequence EPKSCDKTHTCPPCP (SEQ ID NO: 56) or EPKSCDKTHTCPPCPAPELL (SEQ ID NO:57). The Fc region may be from a human IgG4 and at least 5 amino acids are truncated from the C-terminus of the FLT3L extracellular domain. The Fc Region may comprise or further comprise a hinge region. The Fc region may be derived from a human IgG4 isotype and wherein at least 5 amino acids are truncated from the C-terminus of the FLT3L extracellular domain, e.g., wherein the FLT3L extracellular domain does not comprise the amino acid sequence PTAPQ.

The patient or subject may be one that has been previously treated for a condition or indication described herein. The patient or subject may be one that was resistant to the previous treatment. The patient or subject may be one that has been diagnosed with and/or is susceptible to a condition or indication described herein. The method may further comprise administration of an additional therapy, such as, for example, additional therapies described herein.

The terms “protein”, “polypeptide” and “peptide” are used interchangeably herein when referring to a gene product or synthetic amino acid polymer.

The terms “subject,” “mammal,” and “patient” are used interchangeably. In some embodiments, the subject being treated is a mammal. In some embodiments, the subject is a human. In some embodiments, the subject is a mouse, rat, rabbit, dog, donkey, sheep, goat, pig, or a laboratory test animal such as fruit fly, zebrafish, etc.

It is contemplated that the methods and compositions include exclusion of any of the embodiments described herein.

The terms “a” and “an” are defined as one or more unless this disclosure explicitly requires otherwise.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, the methods and systems of the present invention that “comprises,” “has,” “includes” or “contains” one or more elements possesses those one or more elements, but is not limited to possessing only those one or more elements. Likewise, an element of a method or system of the present invention that “comprises,” “has,” “includes” or “contains” one or more features possesses those one or more features, but is not limited to possessing only those one or more features.

The feature or features of one embodiment may be applied to other embodiments, even though not described or illustrated, unless expressly prohibited by this disclosure or the nature of the embodiments. Any peptide or polypeptide described herein may be implemented in the context of any method provided herein, and vice versa, unless explicitly excluded or irrelevant or unworkable. This includes embodiments described in the Examples section.

Any method or system of the present invention can consist of or consist essentially of—rather than comprise/include/contain/have-any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb. A composition “consisting essentially of” the recited elements excludes any further active ingredients but does not exclude pharmaceutical excipients, buffers, structural components, etc.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1B: Expression (A) of and FLT3-binding (B) of FLT3L-MSA.

FIG. 2A-2D: In vivo characterization of activity.

FIG. 3H-3J: Flt3L-MSA gut study-myeloid.

FIG. 4A-4G: Anti-Drug Antibody Prevention.

FIG. 5A-5N. Improving Tolerance Induction by Combining Flt3L-MSA with Oral Immunotherapy.

FIG. 6A-6D: Biomolecular characterization of Flt3L variants: A) SDS-PAGE of Flt3L-MSA and WT Flt3L under Non-reducing and reduction conditions to demonstrate purity of final drug candidate. B) ELISA demonstrating affinity of the materials for the cognate receptor. C) General bioactivity of the variants over time on Flt3L generated BMDCs. D) Concentration dependent bioactivity of the variants as demonstrated by flow cytometry staining of phosphorylated ERK1/2

FIG. 7A-7D: Pharmacokinetics of Flt3L variants. A) schematic representation of the experiment following a single 10 ug injection and bleeds at the indicated timepoints. B) Flt3L presence in the blood as measured via ELISA. C) Area under the curve for 2B demonstrating overall exposure to the cytokines. D) Normalized values to show the kinetics of the 2 drugs with WT Flt3L showing a half life of 10.6 hr and the Flt3L-SA variant having a half-life of 55.8 hours.

FIG. 8A-8C: Pharmacodynamics of Flt3L-SA. A) schematic representation of the experiment. B) Overall DC expansion over time normalized to the saline treated (t=0). C) Treg subsets as a proportion of total CD4 T cells over time normalized to saline treated (t=0).

FIG. 9: schematic representation of the experiment in FIGS. 10-12.

FIG. 10A-10C: Splenic DC characterization after 2 treatments of Flt3L variants. A) DC expansion after treatment. B) Staining for activation markers on splenic DCs after treatment. C) Characterization of splenocytes for expression of the TGF-B precursor protein LAP.

FIG. 11A-11C: Characterization of DCs in the gut dLN. A) DC expansion after treatment with indicated Flt3L variants. B) Staining for activation markers on splenic DCs after treatment. C) Characterization of splenocytes for expression of the TGF-B precursor protein LAP. Each set of three data bars for each condition on the X axis of FIGS. 11A-11C correspond to saline, WT FLT3L, and Flt3L-SA, respectively.

FIG. 12A-12B: Treg quantification and characterization after treatment with Flt3L variants. A) Splenic Treg enumeration and expression of early activation marker PD-1. B) Gut dLN enumeration of Treg presence and expression of early activation marker PD-1 and marker for gut homing (integrin a4b7). Each set of three data bars for each condition on the X axis of FIGS. 12A-12B correspond to saline, WT FLT3L, and Flt3L-SA, respectively.

FIG. 13: Schematic representation of experiment used in FIGS. 14-15

FIG. 14A-14D: Quantification of antibodies present in the serum of treated mice overtime and the determination of states which are likely to cause infusion reactions. A) Total IgG overtime, stars represent significance in comparison to challenge only control. Dotted line represents the limit where mice are likely to develop infusion reactions, as determined in FIG. 14B. B) Cohorts of mice which did or did not show symptomatology of infusion reactions at the final challenge plotted against the IgG titer they had present in serum at the bleed before the challenge. C) Likelihood of seroconversion to an IgG titer of 4 overtime, using a titer of 4 as likely to cause infusion reaction symptomatology as demonstrated in FIG. 14B. D) Following mice for infusion reaction symptoms at each challenge with fractions representing number of mice which did not show symptomatology.

FIG. 15A-15C: Splenic T cell characterization at euthanasia on day 46. A) T follicular helper cell proportion found in the spleen. B) Total Treg presence in the spleen. C) Proportion of T follicular helper cell population displaying a T follicular regulatory phenotype.

FIG. 16A-16N: Co-treatment of Flt3L-SA with oral antigen for oral immunotherapy. A) schematic representation of experiment. B) Quantification of antigen specific CD4+ T cells in the vaccine draining LN. C) Anergy staining in antigen specific CD4+ T cells in the vaccine dLN. D) Quantification of antigen specific Tregs in the vaccine dLN. E) Quantification of TOX+ antigen specific CD4+ T cells in the vaccine dLN. F) Quantification of antigen specific CD8+ T cells in the vaccine dLN. G) Quantification of PD-1 staining on antigen specific CD8+ T cells in the vaccine dLN. H) Quantification of markers of terminal exhaustion on antigen specific CD8+ T cells in the vaccine dLN. I) Quantification of antigen specific CD4+ T cells in spleen. J) Quantification of antigen specific Treg induction in the spleen. K) Quantification of antigen specific CD4+ T cell anergy in spleen. L) Quantification of antigen specific CD8+ T cells in the spleen. M) Quantification of PD-1 staining on antigen specific CD8+ T cells in spleen. N) Quantification of markers of terminal exhaustion on antigen specific CD8+ T cells in spleen.

FIG. 17A-17L: Pre-treatment with Flt3L-SA before addition of antigen-specific cells and oral immunotherapy. A) schematic representation of experiment. B) Quantification of CFSE-diluted (of only FITC positive Va5/VB5 CD4 T cells) in the mesenteric lymph nodes. C) Quantification of CFSE-diluted (of all Va5/VB5 CD4 T cells) in the mesenteric lymph nodes. D) Anergic T cells with the Va2/VB5 TCR, based on cells that stained doubly positive for anergy markers FR4 and CD73. E) Anergic CD4 T cells, based on cells that stained doubly positive for anergy markers FR4 and CD73. F) Corresponding ratio of D) to E) in each sample. G) Quantification of naive CD4 cells (CD44−CD62L+) in mesenteric lymph nodes. H) Quantification of central memory CD4 cells (CD44+CD62L+) in mesenteric lymph nodes. I) Quantification of effector memory CD4 cells (CD44+CD62L−) in mesenteric lymph nodes. J) Quantification of naive Vα2/Vβ5 CD4 cells (CD44−CD62L+) in mesenteric lymph nodes. K) Quantification of central memory Vα2/Vβ5 CD4 cells (CD44+CD62L+) in mesenteric lymph nodes. L) Quantification of effector memory Vα2/Vβ5 CD4 cells (CD44+CD62L−) in mesenteric lymph nodes.

### DETAILED DESCRIPTION

There are different states in which a DC can exist and varying degrees of pro-inflammatory signals or pro-tolerogenic anti-inflammatory signals they can relay to the T cells and surrounding immune milieu. A promising and broadly-applicable strategy to prevent or reverse unwanted immune responses involves delivering signals to the DCs in the tissue which promote the differentiation and survival of DCs with a pro-tolerogenic phenotype, increasing tolerogenic presentation of the antigen to the T cells and preventing or ameliorating downstream inflammation and pathology. Engineered Fms Related Receptor Tyrosine Kinase 3 Ligand (Flt3L) constructs described herein can delivery these anti-inflammatory signals. Flt3L is a chemokine which signals through Flt3 on the surface of DCs to induce proliferation of DCs as well as differentiate hematopoietic stem cells towards a DC fate. It's fusion to serum albumin extends the half-life of the molecule, allowing for increased dose efficacy and the accumulation of pro-tolerogenic DCs in the spleen and lymph nodes. These engineered Flt3L constructs have potential to function as a pre-treatment or co-treatment to prevent the development of anti-drug antibodies.

### I. FLT3L POLYPEPTIDES AND FUSION PROTEINS

### A. Flt3L Proteins

Flt3L polypeptide embodiments include those listed below:

| | | SEQ |
|---|---|---|
| Description | Sequence | ID NO: |
| murine | MTPDCYFSHSPISSNFKVKFRELTDHLLKDYPVTVAVNLQDEKHC | 58 |
| Flt3L | KALWSLFLAQRWIEQLKTVAGSKMQTLLEDVNTEIHFVTSCTFQP | |
| | LPECLRFVQTNISHLLKDTCTQLLALKPCIGKACQNFSRCLEVQC | |
| | QPDSSTLLPPRSPIALEATELPEPRPRQ | |
| Human | MTQDCSFQHSPISSDFAVKIRELSDYLLQDYPVTVASNLQDEELC | 59 |
| Flt3L | GGLWRLVLAQRWMERLKTVAGSKMQGLLERVNTEIHFVTKCAFQP | |
| | PPSCLRFVQTNISRLLQETSEQLVALKPWITRQNFSRCLELQCQP | |
| | DSSTLPPPWSPRPLEATAPTA | |
| Flt3L | TQDCSFQHSPISSDFAVKIRELSDYLLQDYPVTVASNLQDEELCG | 40 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRP | |
| Flt3L | TQDCSFQHSPISSDFAVKIRELSDYLLQDYPVTVASNLQDEELCG | 41 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPL | |
| Flt3L | TQDCSFQHSPISSDFAVKIRELSDYLLQDYPVTVASNLQDEELCG | 42 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPLE | |
| Flt3L | TQDCSFQHSPISSDFAVKIRELSDYLLQDYPVTVASNLQDEELCG | 43 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPLEA | |
| Flt3L | TQDCSFQHSPISSDFAVKIRELSDYLLQDYPVTVASNLQDEELCG | 44 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPLEAT | |
| Flt3L | TQDCSFQHSPISSDFAVKIRELSDYLLQDYPVTVASNLQDEELCG | 45 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPLEATA | |
| Flt3L | TQDCSFQHSPISSDFAVKIRELSDYLLQDYPVTVASNLQDEELCG | 46 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPLEATAP | |
| Flt3L | TQDCSFQHSPISSDFAVKIRELSDYLLQDYPVTVASNLQDEELCG | 47 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPLEATAPT | |
| Flt3L | TQDCSFQHSPISSDFAVKIRELSDYLLQDYPVTVASNLQDEELCG | 48 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPLEATAPTA | |
| Flt3L | TQDCSFQHSPISSDFAVKIRELSDYLLQDYPVTVASNLQDEELCG | 49 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPLEATAPTAP | |
| Flt3L | TQDCSFQHSPISSDFAVKIRELSDYLLQDYPVTVASNLQDEELCG | 50 |
| polypeptide | GLWRLVLAQRWMERLKTVAGSKMQGLLERVNTEIHFVTKCAFQPP | |
| | PSCLRFVQTNISRLLQETSEQLVALKPWITRQNFSRCLELQCQPD | |
| | SSTLPPPWSPRPLEATAPTAPQ | |

### B. Serum Proteins

In some aspects, the polypeptides of the disclosure are further linked to a serum protein. Serum proteins include, for example, albumin, globulin, and fibrinogen. Globulins include alpha 1 globulins, alpha 2 globulins, beta globulins, and gamma globulins. The albumin may be mouse, human, bovine, or any other homologous albumin protein. In some aspects, the albumin comprises human serum albumin, which is encoded by the ALB gene. In some aspects, the albumin comprises mouse albumin.

| | | SEQ |
|---|---|---|
| Description | Sequence | ID NO: |
| Murine | EAHKSEIAHRYNDLGEQHFKGLVLIAFSQYLQKCSYDEHAKLVQEVT | 60 |
| Serum | DFAKTCVADESAANCDKSLHTLFGDKLCAIPNLRENYGELADCCTKQ | |
| Albumin | EPERNECFLQHKDDNPSLPPFERPEAEAMCTSFKENPTTFMGHYLHE | |
| | VARRHPYFYAPELLYYAEQYNEILTQCCAEADKESCLTPKLDGVKEK | |
| | ALVSSVRQRMKCSSMQKFGERAFKAWAVARLSQTFPNADFAEITKLA | |
| | TDLTKVNKECCHGDLLECADDRAELAKYMCENQATISSKLQTCCDKP | |
| | LLKKAHCLSEVEHDTMPADLPAIAADFVEDQEVCKNYAEAKDVFLGT | |
| | FLYEYSRRHPDYSVSLLLRLAKKYEATLEKCCAEANPPACYGTVLAE | |
| | FQPLVEEPKNLVKTNCDLYEKLGEYGFQNAILVRYTQKAPQVSTPTL | |
| | VEAARNLGRVGTKCCTLPEDQRLPCVEDYLSAILNRVCLLHEKTPVS | |
| | EHVTKCCSGSLVERRPCFSALTVDETYVPKEFKAETFTFHSDICTLP | |
| | EKEKQIKKQTALAELVKHKPKATAEQLKTVMDDFAQFLDTCCKAADK | |
| | DTCFSTEGPNLVTRCKDALA | |
| Human | MKWVTFISLLFLFSSAYSRGVFRRDAHKSEVAHRFKDLGEENFKALV | 61 |
| Serum | LIAFAQYLQQCPFEDHVKLVNEVTEFAKTCVADESAENCDKSLHTLF | |
| Albumin | GDKLCTVATLRETYGEMADCCAKQEPERNECFLQHKDDNPNLPRLVR | |
| | PEVDVMCTAFHDNEETFLKKYLYEIARRHPYFYAPELLFFAKRYKAA | |
| | FTECCQAADKAACLLPKLDELRDEGKASSAKQRLKCASLQKFGERAF | |
| | KAWAVARLSQRFPKAEFAEVSKLVTDLTKVHTECCHGDLLECADDRA | |
| | DLAKYICENQDSISSKLKECCEKPLLEKSHCIAEVENDEMPADLPSL | |
| | AADFVESKDVCKNYAEAKDVFLGMFLYEYARRHPDYSVVLLLRLAKT | |
| | YETTLEKCCAAADPHECYAKVFDEFKPLVEEPQNLIKQNCELFEQLG | |
| | EYKFQNALLVRYTKKVPQVSTPTLVEVSRNLGKVGSKCCKHPEAKRM | |
| | PCAEDYLSVVLNQLCVLHEKTPVSDRVTKCCTESLVNRRPCFSALEV | |
| | DETYVPKEFNAETFTFHADICTLSEKERQIKKQTALVELVKHKPKAT | |
| | KEQLKAVMDDFAAFVEKCCKADDKETCFAEEGKKLVAASQAALGL | |

In some aspects, the serum protein comprises a polypeptide of SEQ ID NO:60 or 61, or a fragment thereof, or a polypeptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to SEQ ID NO:60, 61, or a fragment thereof.

### C. Flt3L-Fc Fusion Proteins

Methods of the disclosure employ the use of FLT3L-Fc fusion proteins that have an extended serum half-life in a human subject, relative to soluble FLT3L.

The fusion protein may comprise a human fms related tyrosine kinase 3 ligand (FLT3L) extracellular domain operably linked to an immunoglobulin fragment crystallizable region (Fc region). The fusion protein may have least 5 amino acids are truncated from the C-terminus of the FLT3L extracellular domain. The Fc region may exclude a hinge region.

The FLT3L extracellular domain may be derived from a human FLT3L extracellular domain. The fusion protein may be capable of binding to human FLT3. The FLT3L extracellular domain may be from FLT3L isoform 1 or from FLT3L isoform 2. At least 6, 7, 8, 9, 10, 11, 12, 13, 14 or 15 amino acids may be truncated from the C-terminus of the FLT3L extracellular domain. The FLT3L extracellular domain may exclude (e.g., is deleted, removed or excluded) the amino acid sequence PTAPQ (SEQ ID NO:28), APTAPQ (SEQ ID NO: 29), TAPTAPQ (SEQ ID NO:30), ATAPTAPQ (SEQ ID NO:31), EATAPTAPQ (SEQ ID NO: 32), LEATAPTAPQ (SEQ ID NO:33), PTAPQPP (SEQ ID NO:34), APTAPQPP (SEQ ID NO: 35), TAPTAPQPP (SEQ ID NO:36), ATAPTAPQPP (SEQ ID NO:37), EATAPTAPQPP (SEQ ID NO:38), or LEATAPTAPQPP (SEQ ID NO:39). The FLT3L extracellular domain may comprise or further comprise a N-terminal signal peptide. The FLT3L extracellular domain may comprise or further comprise one or more of the following amino acid substitutions: H8Y; K84E; S102A; and/or S125A; wherein the amino acid residue positions are with reference to SEQ ID NOs: 1-18, 21-27 or 40-50. One or both of serine residues at positions 102 and 125 may be substituted to alanine, wherein the amino acid residue positions are with reference to SEQ ID NOs: 1-18, 21-27 or 40-50.

The Fc region may be from a human IgG1, IgG2, IgG3 or IgG4. The Fc region may be from a human IgG1 or IgG4. The Fc region may comprise a human IgG1 isotype and one or more amino acid substitutions in the Fc region at a residue position selected from the group consisting of: N297A, N297G, N297Q, N297G, D265A, L234A, L235A, C226S, C229S, P238S, E233P, L234V, P238A, A327Q, A327G, P329A, P329G, K322A, L234F, L235E, P331S, T394D, A330L, M252Y, S254T, T256E, M428L, N434S, T366W, T366S, L368A, Y407V, and any combination thereof, wherein the numbering of the residues is according to EU numbering. The Fc region may comprise a human IgG1 isotype and one or more amino acid substitutions in the Fc region at a residue position selected from the group consisting of: L234A, L234V, L234F, L235A, L235E, P331S, and any combination thereof, wherein the numbering of the residues is according to EU numbering. The Fc region may comprise a human IgG4 isotype and one or more amino acid substitutions in the Fc region at a residue position selected from the group consisting of: E233P, F234V, F234A, L235A, G237A, E318A, S228P, L235E, T394D, M252Y, S254T, T256E, N297A, N297G, N297Q, T366W, T366S, L368A, Y407V, M428L, N434S, and any combination thereof, wherein the numbering of the residues is according to EU numbering. The Fc region may comprise a human IgG4 isotype and one or more amino acid substitutions in the Fc region at a residue position selected from the group consisting of: F234V, F234A, L235A, L235E, S228P, and any combination thereof, wherein the numbering of the residues is according to EU numbering. The Fc region may comprise the following amino acids at the indicated positions (EU index numbering): (i) Tyrosine at position 252, threonine at position 254 and glutamic acid at position 256 (YTE); or (ii) Leucine at position 428 and serine at position 434 (LS).

The FLT3L extracellular domain may comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs: 40-50. The Fc region may comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs: 51-55, 62, and 63. The fusion protein may comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs: 1-18 and 21-27.

The Fc region may be from a human IgG1 and may exclude a hinge region. The C-terminus of the FLT3L extracellular domain may not be truncated. The fusion protein may comprise or consist of an amino acid sequence of SEQ ID NO:1. The fusion protein may comprise or consist of an amino acid sequence of SEQ ID NO:9. The fusion protein may comprise or consist of an amino acid sequence selected from the group consisting of SEQ ID NOs: 1, 2, 5, 7, 9, 10, 13, 15, 22, 23 and 24, or comprise or consist of an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1, 2, 5, 7, 9, 10, 13, 15, 22, 23 and 24, wherein the Fc region is derived from a human IgG1 isotype and does not comprise a hinge region, e.g., does not the amino acid sequence EPKSCDKTHTCPPCP (SEQ ID NO:56) or EPKSCDKTHTCPPCPAPELL (SEQ ID NO:57). The Fc region may be from a human IgG4 and at least 5 amino acids may be truncated from the C-terminus of the FLT3L extracellular domain. The Fc region may comprise a hinge region. The fusion protein may comprise or consist of an amino acid sequence of SEQ ID NO:6. The fusion protein may comprise or consist of an amino acid sequence of SEQ ID NO:14. The fusion protein may comprise or consist of an amino acid sequence selected from the group consisting of SEQ ID NOs: 3, 4, 6, 8, 11, 12, 14, 16, 17, 18, 25 and 26, or comprise or consist of an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 3, 4, 6, 8, 11, 12, 14, 16, 17, 18, 25 and 26, wherein the Fc region is derived from a human IgG4 isotype and wherein at least 5 amino acids are truncated from the C-terminus of the FLT3L extracellular domain, e.g., wherein the FLT3L extracellular domain does not comprise the amino acid sequence PTAPQ (SEQ ID NO:28).

The fusion protein may comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs: 19-20.

Fc regions, fusion proteins, and Flt3L polypeptides useful in the disclosure are described below:

| | | SEQ |
|---|---|---|
| Description | Sequence | ID NO: |
| Murine | AKTTPPSVYPLAPGSAAQTNSMVTLGCLVKGYFPEPVTVTWNSGSL | 62 |
| IgG1 Fc | SSGVHTFPAVLQSDLYTLSSSVTVPSSPRPSETVTCNVAHPASSTK | |
| | VDKKIVPRDCGCKPCICTVPEVSSVFIFPPKPKDVLTITLTPKVTC | |
| | VVVDISKDDPEVQFSWFVDDVEVHTAQTQPREEQFNSTFRSVSELP | |
| | IMHQDWLNGKEFKCRVNSAAFPAPIEKTISKTKGRPKAPQVYTIPP | |
| | PKEQMAKDKVSLTCMITDFFPEDITVEWQWNGQPAENYKNTQPIMN | |
| | TNGSYFVYSKLNVQKSNWEAGNTFTCSVLHEGLHNHHTEKSLSHSP | |
| | GK | |
| Human | ASTKGPSVFPLAPSSKSTSGGTAALGCLVKDYFPEPVTVSWNSGAL | 63 |
| IgG1 Fc | TSGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMISR | |
| | TPEVTCVVVDVSHEDPEVKFNWYVDGVEVHNAKTKPREEQYNSTYR | |
| | VVSVLTVLHQDWLNGKEYKCKVSNKALPAPIEKTISKAKGQPREPQ | |
| | VYTLPPSRDELTKNQVSLTCLVKGFYPSDIAVEWESNGQPENNYKT | |
| | TPPVLDSDGSFFLYSKLTVDKSRWQQGNVFSCSVMHEALHNHYTQK | |
| | SLSLSPGK | |
| Fusion | TQDCSFQHSPISSDFAVKIRELSDYLLQDYPVTVASNLQDEELCGG | 1 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAPTAPQGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPS | |
| | REEMTKNQVSLTCLVKGFYPSDIAVEWESNGQPENNYKTTPPVLDS | |
| | DGSFFLYSKLTVDKSRWQQGNVFSCSVMHEALHNHYTQKSLSLSPG | |
| | K | |
| Fusion | TQDCSFQHSPISSDFAVKIRELSDYLLQDYPVTVASNLQDEELCGG | 2 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAGGPSVFLFPPKPKDTLMISRTPEVTCVVVDVS | |
| | HEDPEVKFNWYVDGVEVHNAKTKPREEQYNSTYRVVSVLTVLHQDW | |
| | LNGKEYKCKVSNKALPAPIEKTISKAKGQPREPQVYTLPPSREEMT | |
| | KNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| Fusion | TQDCSFQHSPISSDFAVKIRELSDYLLQDYPVTVASNLQDEELCGG | 3 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAPTAPQESKYGPPCPPCPAPEFEGGPSVFLFPP | |
| | KPKDTLMISRTPEVTCVVVDVSQEDPEVQFNWYVDGVEVHNAKTKP | |
| | REEQFNSTYRVVSVLTVLHQDWLNGKEYKCKVSNKGLPSSIEKTIS | |
| | KAKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVMH | |
| | EALHNHYTQKSLSLSLGK | |
| Fusion | TQDCSFQHSPISSDFAVKIRELSDYLLQDYPVTVASNLQDEELCGG | 4 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAPTAPQESKYGPPCPPCPAPEAAGGPSVFLFPP | |
| | KPKDTLMISRTPEVTCVVVDVSQEDPEVQFNWYVDGVEVHNAKTKP | |
| | REEQFNSTYRVVSVLTVLHQDWLNGKEYKCKVSNKGLPSSIEKTIS | |
| | KAKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVMH | |
| | EALHNHYTQKSLSLSLGK | |
| Fusion | TQDCSFQHSPISSDFAVKIRELSDYLLQDYPVTVASNLQDEELCGG | 5 |
| Protein | LWRLVLAQRWMERLKTVAGSKMQGLLERVNTEIHFVTKCAFQPPPS | |
| | CLRFVQTNIARLLQETSEQLVALKPWITRONFARCLELQCQPDSST | |
| | LPPPWSPRPLEATAPTAPQGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPS | |
| | REEMTKNQVSLTCLVKGFYPSDIAVEWESNGQPENNYKTTPPVLDS | |
| | DGSFFLYSKLTVDKSRWQQGNVFSCSVMHEALHNHYTQKSLSLSPG | |
| | K | |
| Fusion | TQDCSFQHSPISSDFAVKIRELSDYLLQDYPVTVASNLQDEELCGG | 6 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAESKYGPPCPPCPAPEAAGGPSVFLFPPKPKDT | |
| | LMISRTPEVTCVVVDVSQEDPEVQFNWYVDGVEVHNAKTKPREEQF | |
| | NSTYRVVSVLTVLHQDWLNGKEYKCKVSNKGLPSSIEKTISKAKGQ | |
| | PREPQVYTLPPSQEEMTKNQVSLTCLVKGFYPSDIAVEWESNGQPE | |
| | NNYKTTPPVLDSDGSFFLYSRLTVDKSRWQEGNVFSCSVMHEALHN | |
| | HYTQKSLSLSLGK | |
| Fusion | TQDCSFQHSPISSDFAVKIRELSDYLLQDYPVTVASNLQDEELCGG | 7 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPGGPSVFLFPPKPKDTLMISRTPEVTCVVVDVSHEDPE | |
| | VKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVS | |
| | LTCLVKGFYPSDIAVEWESNGQPENNYKTTPPVLDSDGSFFLYSKL | |
| | TVDKSRWQQGNVFSCSVMHEALHNHYTQKSLSLSPGK | |
| Fusion | TQDCSFQHSPISSDFAVKIRELSDYLLQDYPVTVASNLQDEELCGG | 8 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPESKYGPPCPPCPAPEAAGGPSVFLFPPKPKDTLMISR | |
| | TPEVTCVVVDVSQEDPEVQFNWYVDGVEVHNAKTKPREEQFNSTYR | |
| | VVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQK | |
| | SLSLSLGK | |
| Fusion | TQDCSFQHSPISSDFAVKIRELSDYLLQDYPVTVASNLQDEELCGG | 9 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAPTAPQGGPSVFLFPPKPKDTLYITREPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPS | |
| | REEMTKNQVSLTCLVKGFYPSDIAVEWESNGQPENNYKTTPPVLDS | |
| | DGSFFLYSKLTVDKSRWQQGNVFSCSVMHEALHNHYTQKSLSLSPG | |
| | K | |
| Fusion | TQDCSFQHSPISSDFAVKIRELSDYLLQDYPVTVASNLQDEELCGG | 10 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAGGPSVFLFPPKPKDTLYITREPEVTCVVVDVS | |
| | HEDPEVKFNWYVDGVEVHNAKTKPREEQYNSTYRVVSVLTVLHQDW | |
| | LNGKEYKCKVSNKALPAPIEKTISKAKGQPREPQVYTLPPSREEMT | |
| | KNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| Fusion | TQDCSFQHSPISSDFAVKIRELSDYLLQDYPVTVASNLQDEELCGG | 11 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAPTAPQESKYGPPCPPCPAPEFEGGPSVFLFPP | |
| | KPKDTLYITREPEVTCVVVDVSQEDPEVQFNWYVDGVEVHNAKTKP | |
| | REEQFNSTYRVVSVLTVLHQDWLNGKEYKCKVSNKGLPSSIEKTIS | |
| | KAKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVMH | |
| | EALHNHYTQKSLSLSLGK | |
| Fusion | TQDCSFQHSPISSDFAVKIRELSDYLLQDYPVTVASNLQDEELCGG | 12 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAPTAPQESKYGPPCPPCPAPEAAGGPSVFLFPP | |
| | KPKDTLYITREPEVTCVVVDVSQEDPEVQFNWYVDGVEVHNAKTKP | |
| | REEQFNSTYRVVSVLTVLHQDWLNGKEYKCKVSNKGLPSSIEKTIS | |
| | KAKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVMH | |
| | EALHNHYTQKSLSLSLGK | |
| Fusion | TQDCSFQHSPISSDFAVKIRELSDYLLQDYPVTVASNLQDEELCGG | 13 |
| Protein | LWRLVLAQRWMERLKTVAGSKMQGLLERVNTEIHFVTKCAFQPPPS | |
| | CLRFVQTNIARLLQETSEQLVALKPWITRONFARCLELQCQPDSST | |
| | LPPPWSPRPLEATAPTAPQGGPSVFLFPPKPKDTLYITREPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPS | |
| | REEMTKNQVSLTCLVKGFYPSDIAVEWESNGQPENNYKTTPPVLDS | |
| | DGSFFLYSKLTVDKSRWQQGNVFSCSVMHEALHNHYTQKSLSLSPG | |
| | K | |
| Fusion | TQDCSFQHSPISSDFAVKIRELSDYLLQDYPVTVASNLQDEELCGG | 14 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAESKYGPPCPPCPAPEAAGGPSVFLFPPKPKDT | |
| | LYITREPEVTCVVVDVSQEDPEVQFNWYVDGVEVHNAKTKPREEQF | |
| | NSTYRVVSVLTVLHQDWLNGKEYKCKVSNKGLPSSIEKTISKAKGQ | |
| | PREPQVYTLPPSQEEMTKNQVSLTCLVKGFYPSDIAVEWESNGQPE | |
| | NNYKTTPPVLDSDGSFFLYSRLTVDKSRWQEGNVFSCSVMHEALHN | |
| | HYTQKSLSLSLGK | |
| Fusion | TQDCSFQHSPISSDFAVKIRELSDYLLQDYPVTVASNLQDEELCGG | 15 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPGGPSVFLFPPKPKDTLYITREPEVTCVVVDVSHEDPE | |
| | VKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVS | |
| | LTCLVKGFYPSDIAVEWESNGQPENNYKTTPPVLDSDGSFFLYSKL | |
| | TVDKSRWQQGNVFSCSVMHEALHNHYTQKSLSLSPGK | |
| Fusion | TQDCSFQHSPISSDFAVKIRELSDYLLQDYPVTVASNLQDEELCGG | 16 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPESKYGPPCPPCPAPEAAGGPSVFLFPPKPKDTLYITR | |
| | EPEVTCVVVDVSQEDPEVQFNWYVDGVEVHNAKTKPREEQFNSTYR | |
| | VVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQK | |
| | SLSLSLGK | |
| Fusion | TQDCSFQHSPISSDFAVKIRELSDYLLQDYPVTVASNLQDEELCGG | 17 |
| Protein | LWRLVLAQRWMERLKTVAGSKMQGLLERVNTEIHFVTKCAFQPPPS | |
| | CLRFVQTNIARLLQETSEQLVALKPWITRQNFARCLELQCQPDSST | |
| | LPPPWSPRPESKYGPPCPPCPAPEAAGGPSVFLFPPKPKDTLMISR | |
| | TPEVTCVVVDVSQEDPEVQFNWYVDGVEVHNAKTKPREEQFNSTYR | |
| | VVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQK | |
| | SLSLSLGK | |
| Fusion | TQDCSFQHSPISSDFAVKIRELSDYLLQDYPVTVASNLQDEELCGG | 18 |
| Protein | LWRLVLAQRWMERLKTVAGSKMQGLLERVNTEIHFVTKCAFQPPPS | |
| | CLRFVQTNIARLLQETSEQLVALKPWITRONFARCLELQCQPDSST | |
| | LPPPWSPRPESKYGPPCPPCPAPEAAGGPSVFLFPPKPKDTLYITR | |
| | EPEVTCVVVDVSQEDPEVQFNWYVDGVEVHNAKTKPREEQFNSTYR | |
| | VVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQK | |
| | SLSLSLGK | |
| Fusion | TPDCYFSHSPISSNFKVKFRELTDHLLKDYPVTVAVNLQDEKHCKA | 19 |
| Protein | LWSLFLAQRWIEQLKTVAGSKMQTLLEDVNTEIHFVTSCTFQPLPE | |
| | CLRFVQTNISHLLKDTCTQLLALKPCIGKACQNFSRCLEVQCQPDS | |
| | STLLPPRSPIALEATELPEPRGPTIKPCPPCKCPAPNAAGGPSVFI | |
| | FPPKIKDVLMISLSPIVTCVVVDVSEDDPDVQISWFVNNVEVHTAQ | |
| | TQTHREDYNSTLRVVSALPIQHQDWMSGKEFKCKVNNKDLGAPIER | |
| | TISKPKGSVRAPQVYVLPPPEEEMTKKQVTLTCMVTDFMPEDIYVE | |
| | WTNNGKTELNYKNTEPVLDSDGSYFMYSKLRVEKKNWVERNSYSCS | |
| | VVHEGLHNHHTTKSFSRTPGK | |
| Fusion | TPDCYFSHSPISSNFKVKFRELTDHLLKDYPVTVAVNLQDEKHCKA | 20 |
| Protein | LWSLFLAQRWIEQLKTVAGSKMQTLLEDVNTEIHFVTSCTFQPLPE | |
| | CLRFVQTNISHLLKDTSTQLLALKPCIGKACQNFSRCLEVQCQPDS | |
| | STLLPPRSPIALEATELPEPRGPTIKPCPPCKCPAPNAAGGPSVFI | |
| | FPPKIKDVLMISLSPIVTCVVVDVSEDDPDVQISWFVNNVEVHTAQ | |
| | TQTHREDYNSTLRVVSALPIQHQDWMSGKEFKCKVNNKDLGAPIER | |
| | TISKPKGSVRAPQVYVLPPPEEEMTKKQVTLTCMVTDFMPEDIYVE | |
| | WTNNGKTELNYKNTEPVLDSDGSYFMYSKLRVEKKNWVERNSYSCS | |
| | VVHEGLHNHHTTKSFSRTPGK | |
| Fusion | TQDCSFQHSPISSDFAVKIRELSDYLLQDYPVTVASNLQDEELCGG | 21 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAPTAPQGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTKPPS | |
| | REEMTKNQVSLSCLVKGFYPSDIAVEWESNGQPENNYKTTVPVLDS | |
| | DGSFRLASYLTVDKSRWQQGNVFSCSVMHEALHNHYTQKSLSLSPG | |
| | K | |
| Fusion | TQDCSFQYSPISSDFAVKIRELSDYLLQDYPVTVASNLQDEELCGG | 22 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDSST | |
| | LPPPWSPRPLEATAPTAPQGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPS | |
| | REEMTKNQVSLTCLVKGFYPSDIAVEWESNGQPENNYKTTPPVLDS | |
| | DGSFFLYSKLTVDKSRWQQGNVFSCSVMHEALHNHYTQKSLSLSPG | |
| | K | |
| Fusion | TQDCSFQHSPISSDFAVKIRELSDYLLQDYPVTVASNLQDEELCGG | 23 |
| Protein | LWRLVLAQRWMERLKTVAGSKMQGLLERVNTEIHFVTECAFQPPPS | |
| | CLRFVQTNISRLLQETSEQLVALKPWITRQNFSRCLELQCQPDSST | |
| | LPPPWSPRPLEATAPTAPQGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPS | |
| | REEMTKNQVSLTCLVKGFYPSDIAVEWESNGQPENNYKTTPPVLDS | |
| | DGSFFLYSKLTVDKSRWQQGNVFSCSVMHEALHNHYTQKSLSLSPG | |
| | K | |
| Fusion | TQDCSFQYSPISSDFAVKIRELSDYLLQDYPVTVASNLQDEELCGG | 24 |
| Protein | LWRLVLAQRWMERLKTVAGSKMQGLLERVNTEIHFVTECAFQPPPS | |
| | CLRFVQTNISRLLQETSEQLVALKPWITRQNFSRCLELQCQPDSST | |
| | LPPPWSPRPLEATAPTAPQGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPS | |
| | REEMTKNQVSLTCLVKGFYPSDIAVEWESNGQPENNYKTTPPVLDS | |
| | DGSFFLYSKLTVDKSRWQQGNVFSCSVMHEALHNHYTQKSLSLSPG | |
| | K | |
| Fusion | TQDCSFQHSPISSDFAVKIRELSDYLLQDYPVTVASNLQDEELCGG | 25 |
| Protein | LWRLVLAQRWMERLKTVAGSKMQGLLERVNTEIHFVTKCAFQPPPS | |
| | CLRFVQTNIARLLQETSEQLVALKPWITRQNFARCLELQCQPDSST | |
| | LPPPWSPRPLEATAESKYGPPCPPCPAPEAAGGPSVFLFPPKPKDT | |
| | LYITREPEVTCVVVDVSQEDPEVQFNWYVDGVEVHNAKTKPREEQF | |
| | NSTYRVVSVLTVLHQDWLNGKEYKCKVSNKGLPSSIEKTISKAKGQ | |
| | PREPQVYTLPPSQEEMTKNQVSLTCLVKGFYPSDIAVEWESNGQPE | |
| | NNYKTTPPVLDSDGSFFLYSRLTVDKSRWQEGNVFSCSVMHEALHN | |
| | HYTQKSLSLSLGK | |
| Fusion | TQDCSFQHSPISSDFAVKIRELSDYLLQDYPVTVASNLQDEELCGG | 26 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDAAA | |
| | LPPPWSPRPLEATAESKYGPPCPPCPAPEAAGGPSVFLFPPKPKDT | |
| | LYITREPEVTCVVVDVSQEDPEVQFNWYVDGVEVHNAKTKPREEQF | |
| | NSTYRVVSVLTVLHQDWLNGKEYKCKVSNKGLPSSIEKTISKAKGQ | |
| | PREPQVYTLPPSQEEMTKNQVSLTCLVKGFYPSDIAVEWESNGQPE | |
| | NNYKTTPPVLDSDGSFFLYSRLTVDKSRWQEGNVFSCSVMHEALHN | |
| | HYTQKSLSLSLGK | |
| Fusion | TQDCSFQHSPISSDFAVKIRELSDYLLQDYPVTVASNLQDEELCGG | 27 |
| Protein | LWRLVLAQRWMERLKTVAGSKMQGLLERVNTEIHFVTKCAFQPPPS | |
| | CLRFVQTNISRLLQETSEQLVALKPWITRQNFSRCLELQCQPDAAA | |
| | LPPPWAPRPLEATAEAKYGPPCPPCPAPEAAGGPSVFLFPPKPKDT | |
| | LYITREPEVTCVVVDVSQEDPEVQFNWYVDGVEVHNAKTKPREEQF | |
| | NSTYRVVSVLTVLHQDWLNGKEYKCKVSNKGLPSSIEKTISKAKGQ | |
| | PREPQVYTLPPSQEEMTKNQVSLTCLVKGFYPSDIAVEWESNGQPE | |
| | NNYKTTPPVLDSDGSFFLYSRLTVDKSRWQEGNVFSCSVMHEALHN | |
| | HYTQKSLSLSLGK | |
| Fc region | GGPSVFLFPPKPKDTLMISRTPEVTCVVVDVSHEDPEVKFNWYVDG | 51 |
| | VEVHNAKTKPREEQYNSTYRVVSVLTVLHQDWLNGKEYKCKVSNKA | |
| | LPAPIEKTISKAKGQPREPQVYTLPPSREEMTKNQVSLTCLVKGFY | |
| | PSDIAVEWESNGQPENNYKTTPPVLDSDGSFFLYSKLTVDKSRWQQ | |
| | GNVFSCSVMHEALHNHYTQKSLSLSPGK | |
| Fc region | GGPSVFLFPPKPKDTLYITREPEVTCVVVDVSHEDPEVKFNWYVDG | 52 |
| | VEVHNAKTKPREEQYNSTYRVVSVLTVLHQDWLNGKEYKCKVSNKA | |
| | LPAPIEKTISKAKGQPREPQVYTLPPSREEMTKNQVSLTCLVKGFY | |
| | PSDIAVEWESNGQPENNYKTTPPVLDSDGSFFLYSKLTVDKSRWQQ | |
| | GNVFSCSVMHEALHNHYTQKSLSLSPGK | |
| Fc region | ESKYGPPCPPCPAPEFEGGPSVFLFPPKPKDTLMISRTPEVTCVVV | 53 |
| | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| Fc region | ESKYGPPCPPCPAPEAAGGPSVFLFPPKPKDTLMISRTPEVTCVVV | 54 |
| | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| Fc region | ESKYGPPCPPCPAPEFEGGPSVFLFPPKPKDTLYITREPEVTCVVV | 55 |
| | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |

### II. POLYPEPTIDES

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise or may exclude, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include or exclude 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs: 1-72.

In some embodiments, the protein or polypeptide may comprise or exclude amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs: 1-72.

The protein, polypeptide, or nucleic acid may comprise or exclude 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs: 1-72.

In some embodiments, the polypeptide, protein, or nucleic acid may exclude or comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs: 1-72 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs: 1-72.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOs: 1-72 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOs: 1-72.

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 (or any derivable range therein) of any of SEQ ID NOs: 1-72 and may be a substitution with any amino acid or may be a substitution with an alanine, arginine, asparagine, aspartic acid, cysteine, glutamine, glutamic acid, glycine, histidine, isoleucine, leusine, lysine, methionine, phenylilacnine, proline, serine, threonine, tryptophan, tyrosine, or valine.

Any of SEQ ID NOs: 1-72 may be excluded in the methods and compositions of the disclosure.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylilacnine to tyrosine, leucine or methionine; serine to threonine; threonine to serine; tryptophan to tyrosine; tyrosine to tryptophan or phenylilacnine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### B. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylilacnine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the present disclosure, those that are within ±1 are included, and in other aspects of the present disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylilacnine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### III. NUCLEIC ACIDS

In certain embodiments, the current disclosure concerns recombinant polynucleotides encoding the proteins, polypeptides, and peptides of the disclosure.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including all values and ranges there between, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

In particular embodiments, the invention concerns isolated nucleic acid segments and recombinant vectors incorporating nucleic acid sequences that encode a polypeptide or peptide of the disclosure. The term “recombinant” may be used in conjunction with a polynucleotide or polypeptide and generally refers to a polypeptide or polynucleotide produced and/or manipulated in vitro or that is a replication product of such a molecule.

In other embodiments, the invention concerns isolated nucleic acid segments and recombinant vectors incorporating nucleic acid sequences that encode a polypeptide or peptide of the disclosure.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, the current disclosure provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides.

### A. Vectors

Polypeptides of the disclosure may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding a polypeptide of the disclosure, the vector can encode other polypeptide sequences such as a one or more other bacterial peptide, a tag, or an immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. In some embodiments, the vector comprises pSeqTag-A or pcDNA3.1.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### B. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Various elements/promoters may be employed in the context of the present invention to regulate the expression of a gene. Examples of such inducible elements, which are regions of a nucleic acid sequence that can be activated in response to a specific stimulus, include but are not limited to Immunoglobulin Heavy Chain (Banerji et al., 1983; Gilles et al., 1983; Grosschedl et al., 1985; Atchinson et al., 1986, 1987; Imler et al., 1987; Weinberger et al., 1984; Kiledjian et al., 1988; Porton et al.; 1990), Immunoglobulin Light Chain (Queen et al., 1983; Picard et al., 1984), T Cell Receptor (Luria et al., 1987; Winoto et al., 1989; Redondo et al.; 1990), HLA DQ and/or DQ (Sullivan et al., 1987), γ Interferon (Goodbourn et al., 1986; Fujita et al., 1987; Goodbourn et al., 1988), Interleukin-2 (Greene et al., 1989), Interleukin-2 Receptor (Greene et al., 1989; Lin et al., 1990), MHC Class II 5 (Koch et al., 1989), MHC Class II HLA-DR (Sherman et al., 1989), β-Actin (Kawamoto et al., 1988; Ng et al.; 1989), Muscle Creatine Kinase (MCK) (Jaynes et al., 1988; Horlick et al., 1989; Johnson et al., 1989), Prealbumin (Transthyretin) (Costa et al., 1988), Elastase I (Ornitz et al., 1987), Metallothionein (MTII) (Karin et al., 1987; Culotta et al., 1989), Collagenase (Pinkert et al., 1987; Angel et al., 1987), Albumin (Pinkert et al., 1987; Tronche et al., 1989, 1990), □-Fetoprotein (Godbout et al., 1988; Campere et al., 1989), γ-Globin (Bodine et al., 1987; Perez-Stable et al., 1990), □-Globin (Trudel et al., 1987), c-fos (Cohen et al., 1987), c-Ha-Ras (Triesman, 1986; Deschamps et al., 1985), Insulin (Edlund et al., 1985), Neural Cell Adhesion Molecule (NCAM) (Hirsh et al., 1990), □1-Antitrypain (Latimer et al., 1990), H2B (TH2B) Histone (Hwang et al., 1990), Mouse and/or Type I Collagen (Ripe et al., 1989), Glucose-Regulated Proteins (GRP94 and GRP78) (Chang et al., 1989), Rat Growth Hormone (Larsen et al., 1986), Human Serum Amyloid A (SAA) (Edbrooke et al., 1989), Troponin I (TN I) (Yutzey et al., 1989), Platelet-Derived Growth Factor (PDGF) (Pech et al., 1989), Duchenne Muscular Dystrophy (Klamut et al., 1990), SV40 (Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988), Polyoma (Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell et al., 1988), Retroviruses (Kriegler et al., 1982, 1983; Levinson et al., 1982; Kriegler et al., 1983, 1984a, b, 1988; Bosze et al., 1986; Miksicek et al., 1986; Celander et al., 1987; Thiesen et al., 1988; Celander et al., 1988; Choi et al., 1988; Reisman et al., 1989), Papilloma Virus (Campo et al., 1983; Lusky et al., 1983; Spandidos and Wilkie, 1983; Spalholz et al., 1985; Lusky et al., 1986; Cripe et al., 1987; Gloss et al., 1987; Hirochika et al., 1987; Stephens et al., 1987), Hepatitis B Virus (Bulla et al., 1986; Jameel et al., 1986; Shaul et al., 1987; Spandau et al., 1988; Vannice et al., 1988), Human Immunodeficiency Virus (Muesing et al., 1987; Hauber et al., 1988; Jakobovits et al., 1988; Feng et al., 1988; Takebe et al., 1988; Rosen et al., 1988; Berkhout et al., 1989; Laspia et al., 1989; Sharp et al., 1989; Braddock et al., 1989), Cytomegalovirus (CMV) IE (Weber et al., 1984; Boshart et al., 1985; Foecking et al., 1986), Gibbon Ape Leukemia Virus (Holbrook et al., 1987; Quinn et al., 1989).

Inducible elements include, but are not limited to MT II-Phorbol Ester (TFA)/Heavy metals (Palmiter et al., 1982; Haslinger et al., 1985; Searle et al., 1985; Stuart et al., 1985; Imagawa et al., 1987, Karin et al., 1987; Angel et al., 1987b; McNeall et al., 1989); MMTV (mouse mammary tumor virus)—Glucocorticoids (Huang et al., 1981; Lee et al., 1981; Majors et al., 1983; Chandler et al., 1983; Lee et al., 1984; Ponta et al., 1985; Sakai et al., 1988); γ-Interferon—poly(rI)x/poly(rc) (Tavernier et al., 1983); Adenovirus 5 E2—EIA (Imperiale et al., 1984); Collagenase—Phorbol Ester (TPA) (Angel et al., 1987a); Stromelysin—Phorbol Ester (TPA) (Angel et al., 1987b); SV40-Phorbol Ester (TPA) (Angel et al., 1987b); Murine MX Gene—Interferon, Newcastle Disease Virus (Hug et al., 1988); GRP78 Gene—A23187 (Resendez et al., 1988); β-2-Macroglobulin-IL-6 (Kunz et al., 1989); Vimentin—Serum (Rittling et al., 1989); MHC Class I Gene H-2b—Interferon (Blanar et al., 1989); HSP70—EIA/SV40 Large T Antigen (Taylor et al., 1989, 1990a, 1990b); Proliferin-Phorbol Ester/TPA (Mordacq et al., 1989); Tumor Necrosis Factor-PMA (Hensel et al., 1989); and Thyroid Stimulating Hormone Gene-Thyroid Hormone (Chatterjee et al., 1989).

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

### C. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

### D. Selectable and Screenable Markers

In certain embodiments of the invention, cells containing a nucleic acid construct of the current disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. As an alternative, 2A peptides could be used to introduce ribosomal skips to enable expression of multiple polypeptidic or protein sequences.

### E. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL□ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### IV. COMPOSITIONS

In some embodiments, pharmaceutical compositions are administered to a subject. Different aspects involve administering an effective amount of a composition to a subject. In some embodiments, a composition comprising a peptide of the disclosure may be administered to the subject or patient to treat pathogenic immune responses. Additionally, such compositions can be administered in combination with an additional therapy.

### A. Carriers and Excipients

Pharmaceutically acceptable carriers or excipients may be used to deliver embodiments as described herein. Excipient refers to an inert substance used as a diluent or vehicle for a therapeutic agent. Pharmaceutically acceptable carriers are used, in general, with a compound (eg. peptide of the disclosure) so as to make the compound useful for a therapy or as a product. In general, for any substance, a carrier is a material that is combined with the substance for delivery to an animal. Conventional pharmaceutical carriers, aqueous, powder or oily bases, thickeners and the like may be necessary or desirable. In some cases the carrier is essential for delivery, e.g., to solubilize an insoluble compound for liquid delivery; a buffer for control of the pH of the substance to preserve its activity; or a diluent to prevent loss of the substance in the storage vessel. In other cases, however, the carrier is for convenience, e.g., a liquid for more convenient administration. Pharmaceutically acceptable salts of the compounds described herein may be synthesized according to methods known to those skilled in the arts. Thus a pharmaceutically acceptable compositions are highly purified to be free of contaminants, are sterile, biocompatible and not toxic, and further may include a carrier, salt, or excipient suited to administration to a patient. In the case of water as the carrier, the water is highly purified and processed to be free of contaminants, e.g., endotoxins.

The compounds described herein may be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. Thus the deliverable compound may be made in a form suitable for oral, rectal, topical, intravenous injection, intra-articular injection, intradermal, intramuscular, and/or parenteral administration. Carriers include solids or liquids, and the type of carrier is chosen based on the type of administration being used. Suitable binders, lubricants, disintegrating agents, coloring agents, flavoring agents, flow-inducing agents, and melting agents may be included as carriers, e.g., for pills. For instance, an active component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. The compounds can be administered orally in solid dosage forms, such as capsules, tablets, and powders, or in liquid dosage forms, such as elixirs, syrups, and suspensions. The active compounds can also be administered parentally, in sterile liquid dosage forms. Buffers for achieving a physiological pH or osmolarity may also be used.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

### B. Dosage

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or ug/kg or micrograms/kg/minute or mg/kg/min or micrograms/kg/hour or mg/kg/hour, or any range derivable therein of an agent of the disclosure (e.g. growth factor, cytokine, peptide, polypeptide, functional moiety, etc. . . . ).

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after symptoms of an infection have disappeared or been reduced.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Antigen Tolerance Induction Through Use of FLT3L Variants

Antibody, enzyme replacement, and gene therapies have demonstrated efficacy in treating a range of diseases, and biologics such as these accounted for 20% of the FDA approvals in 2020 (1). However, these classes of therapeutics, that are composed of proteins not native to the patient's body such as engineered antibodies, enzymes in patients lacking those genes, or viruses used to delivery genetic material, are known to trigger an immune response in those patients. These immune responses include both T cand B cell responses and result in side-effects ranging from hypersensitivity reactions upon administration, to life-threatening anaphylaxis (2-4). Furthermore, since antibody responses are very common against these proteins, antibody recognition and clearance of the therapeutic can reduce or eliminate efficacy (5-7). Dose escalation from small more tolerable doses up to larger therapeutic doses is used in very immunogenic therapies to enable patients to tolerate the therapeutic and preserve its efficacy (8). However, this method is only effective for approximately 65% of patients (9). Engineered Flt3L constructs can be used to significantly increase the efficacy and safety of this tolerance scheme for any therapeutic antigen via simple co-administration to promote the development and accumulation of tolerogenic DCs during this immune priming phase. Thus, shown herein is how pretreatment with Flt3L constructs, coupled with low doses of the immunogenic protein, resulted in the lasting reduction of a subject's anti-drug antibody responses. In addition, shown herein are means to exploit the tolerogenic capacity of the oral mucosa, for example, by adding Flt3L fused to Mouse Serum Albumin (MSA; Flt3L-MSA) to the therapeutic regimen to induce antigen-specific tolerogenic T cell responses following challenge at a non-oral site.

### A. Results

### 1. General Molecule Production and Characterization:

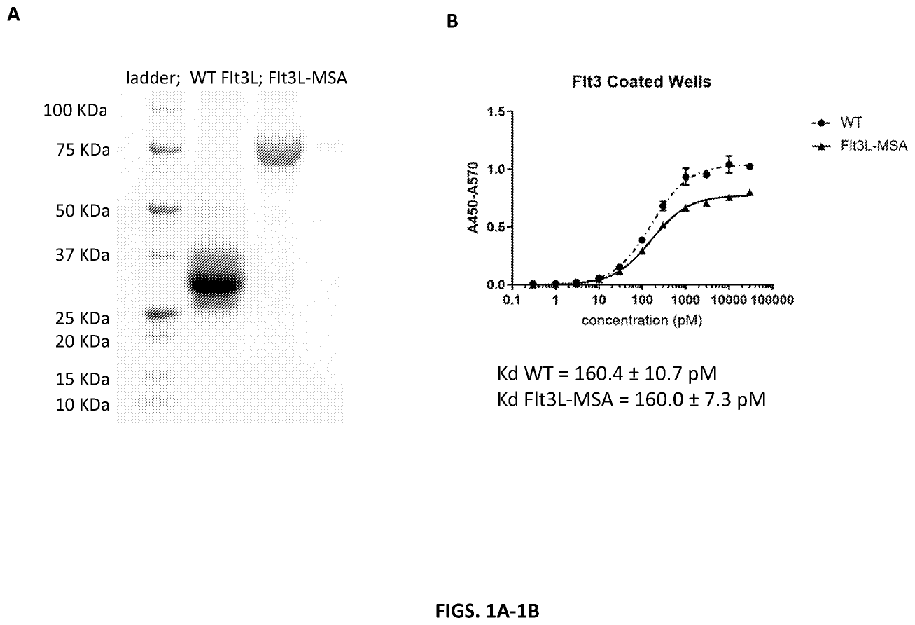

Protein was cloned, produced, and purified as described in methods. Following purification, bands were confirmed at the expected sizes via SDS-PAGE under reducing conditions (WT at around 30 KDa with multiple bands due to glycosylation differences and Flt3L-MSA at around 75 KDa). Binding of each protein to the receptor for Flt3L (murine Flt3, R&D 768-F3-050) was then confirmed via ELISA. Briefly, high binding plates were coated overnight with 50 μL of 1 nM protein in a Carb/Bicarb buffer pH of 9. The plates were then washed and blocked using R&D blocking buffer before the addition of Flt3L variants at different concentrations in blocking buffer. Detection occurred using biotinylated αFlt3L and subsequent incubation with avidin-HRP. Binding was then visualized via incubation with TMB solution until highest concentrations reached an approximate absorbance of 1 and the reaction was stopped using 2N H2SO4. The binding of both WT and MSA fused Flt3L was determined to be equivalent with a KD of 160 pM (FIG. 1A-B).

### 2. In Vivo Characterization of Activity

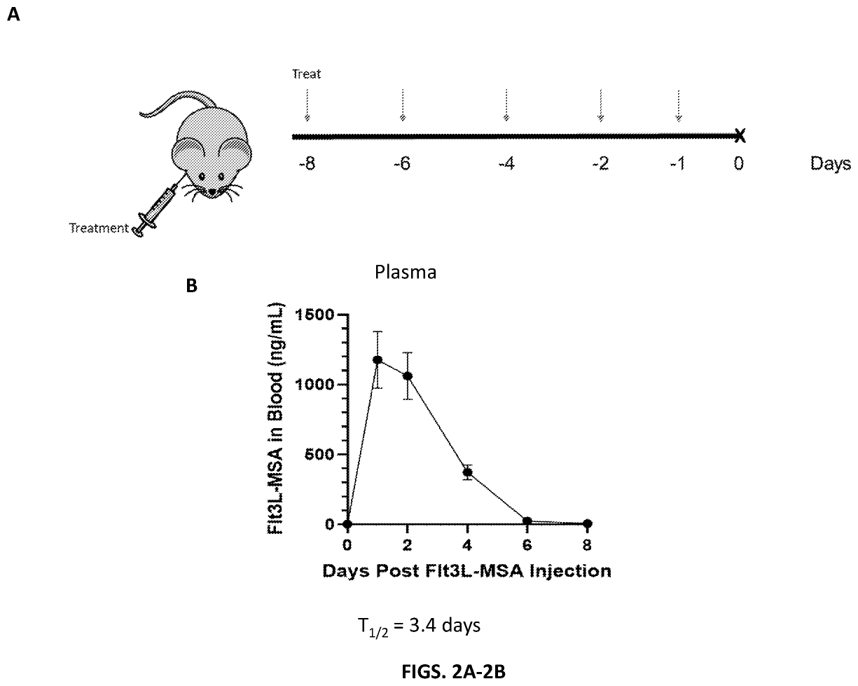

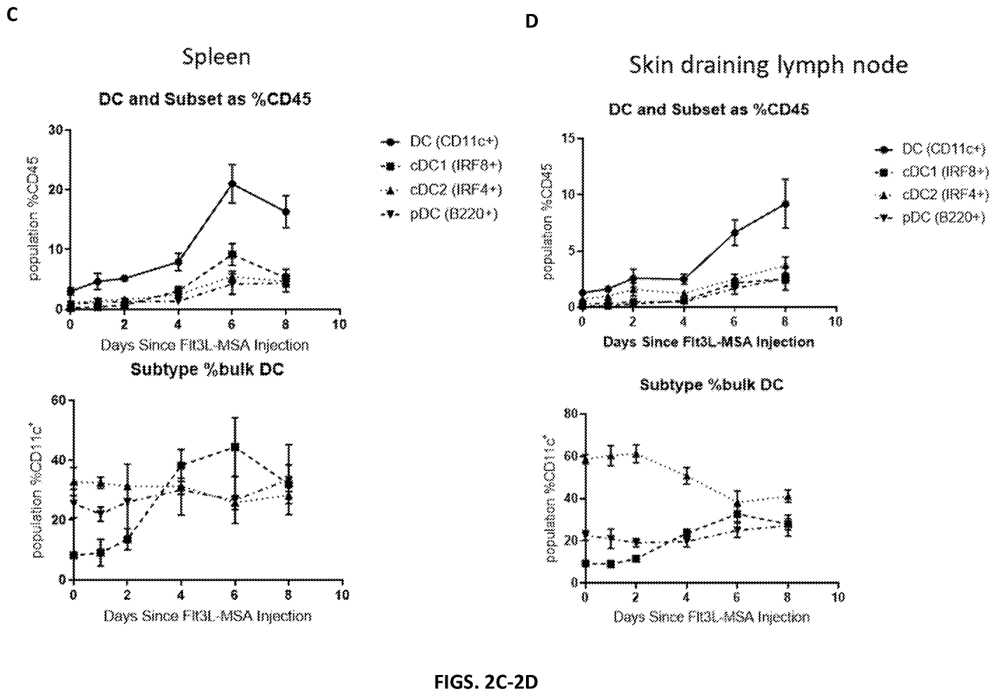

Once the molecules were produced and binding confirmed, the in vivo pharmacokinetics and activity of the fusion protein was determined. To do so, 9 week-old C57BL/6 mice were treated at a given timepoint ranging from 8 days to 1 day prior to euthanasia with 10 ug Flt3L basis (44 μg total protein content) as a single subcutaneous injection in the forelimb (FIG. 2A). At time of euthanasia, blood, spleen, and the axillary and brachial lymph nodes draining the injection site were harvested. Flt3L-MSA concentration in the plasma was then calculated via ELISA against a Flt3L-MSA standard. From this, the serum half-life of Flt3L-MSA was found to be roughly 3.4 days (FIG. 2B). As Flt3L is a major cytokine involved in the development of dendritic cells, the dendritic cell subsets in the lymphoid organs were then characterized via flow cytometry. In the spleen, a slow rise in CD11c+ DC populations was observed until 4 days following injection, and then a sudden sharp rise in populations on day 6 was observed, these results suggested de novo DC differentiation from the bone marrow rather than peripheral proliferation (FIG. 2 C, top panel). Most notably, a major increase in the IRF8+ Type 1 conventional DC (cDC1) compartment was observed when compared to untreated mice; by day 6, cDC1s comprise roughly 40% of the total DC compartment, while they only accounted for about 10% of the compartment in untreated mouse spleens. Also to note, while all DC compartments (cDC1, cDC2, and pDC) increased in similar trends when normalized to total CD45+ cells, when normalized as a percentage of CD11c+ cells, only the cDC1 compartment showed notable increases in the spleen, suggesting a preferred skewing to the cDC1 fate (FIG. 2 C, bottom panel). Similar trends were also seen in the injection draining lymph nodes with a slow rise in DC populations until day 4, after which the populations all experienced a sharp rise when plotted as a percent of total CD45+ cells. Furthermore, just like in the spleen, only the cDC1 population showed a notable increase over time when reported as percent of the total DC compartment, whereas the pDC percentage remained stable at about 20%, and the cDC2 compartment decreased over time in coordination with the increase of cDC1s (FIG. 2 D). Taken together, these data further suggested de novo DC proliferation from the bone marrow rather than localized DC proliferation.

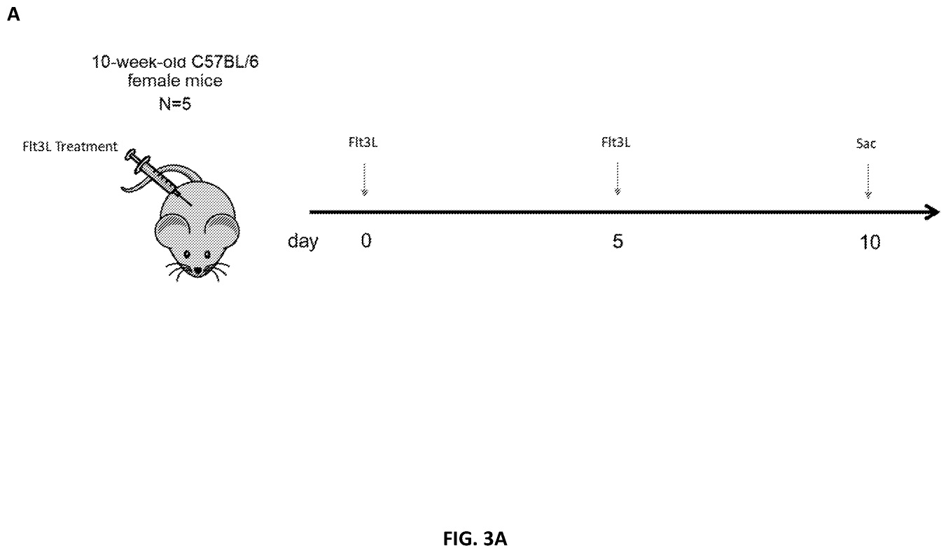

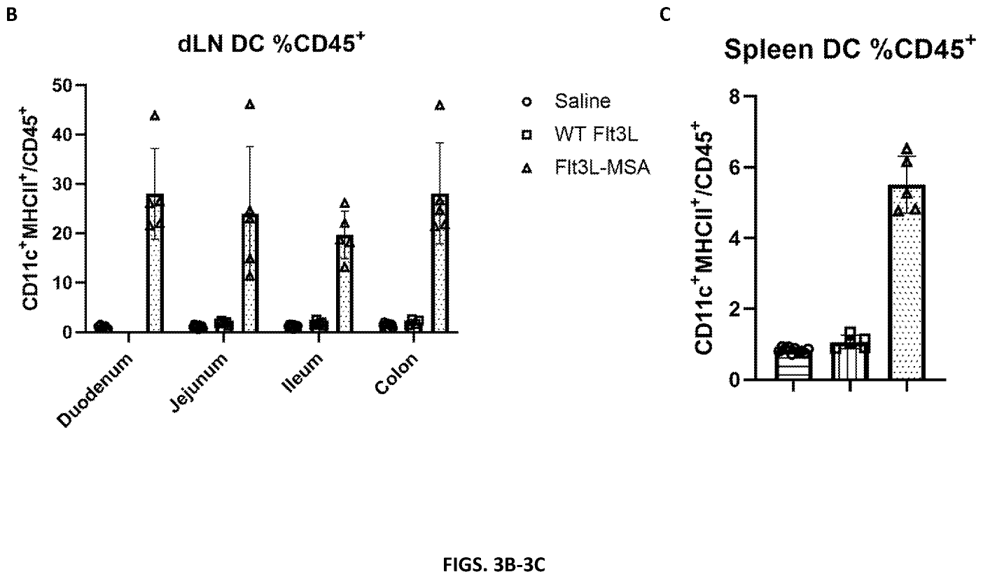

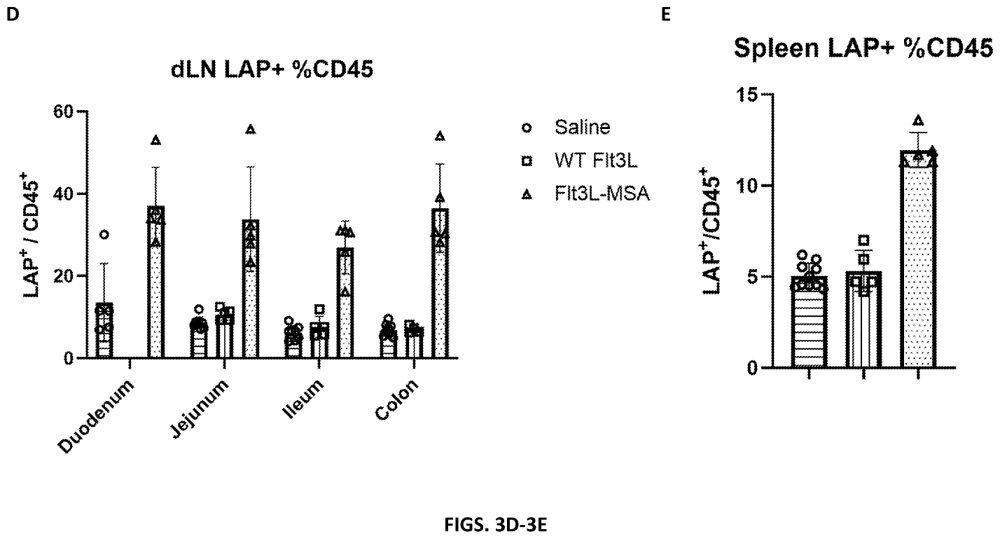

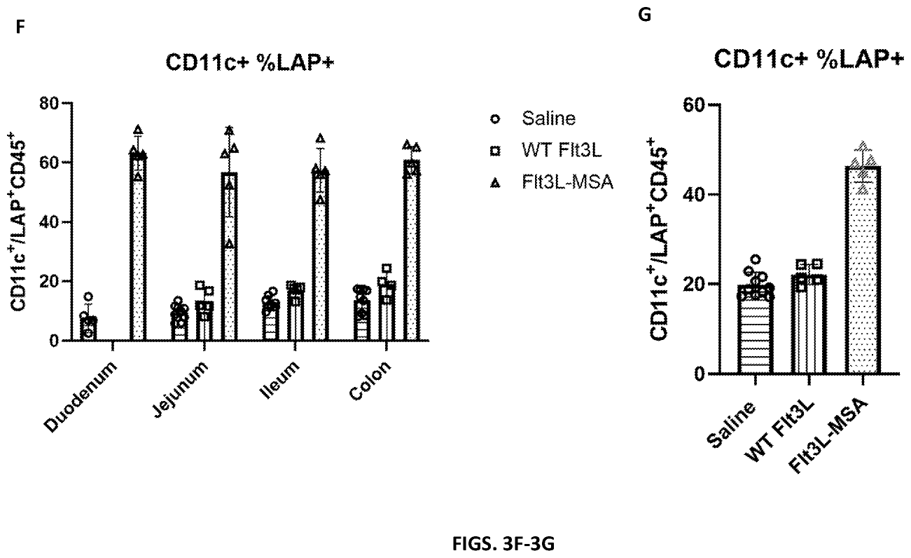

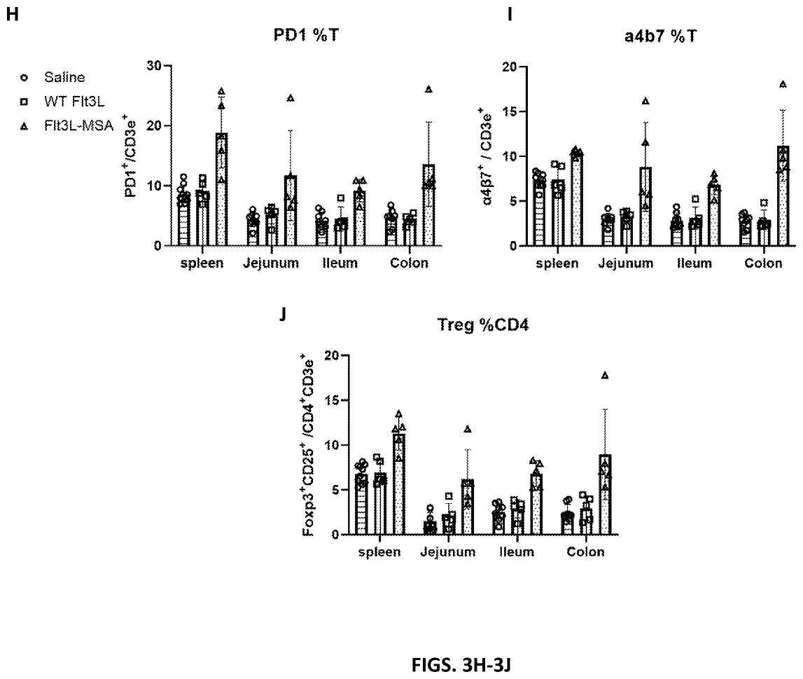

In the aforementioned study, a large increase in the size of the mesenteric draining lymph nodes was observed, which prompted the inventors to further investigate the phenotypes present in those sites following a treatment regimen. Mice were treated with either saline, 10 μg wild type Flt3L, or 10 μg molar equivalent Flt3L-MSA s.c. 5 days apart for a total of 2 doses, and euthanized 5 days after the final dose. As in the previous study, mice treated with Flt3L-MSA showed a significant increase in the percentage of the hematopoietic compartment as compared to saline and/or WT treated mice; these differences were much more pronounced in the gut draining lymph nodes where the lymph nodes from Flt3L-MSA treated mice showed DCs comprising about 20% of the compartment, this was in stark difference to DCs comprising ˜1% of the compartment in saline and/or WT treated mice (FIG. 3A-C). Due to its role in Treg differentiation and generally in immunologic suppression as well as previously published data showing that Flt3L treatment leads to Treg proliferation, the inventors then sought to determine if Transforming Growth Factor Beta (TGF-β) expression was also different between treatment groups. Here too, large increases in the percentage of cells expressing the TGF-β precursor, Latent Associated Peptide (LAP), were observed, but only in the engineered cytokine group, where double the percent of CD45+ cells staining positive for LAP were observed (FIG. 3D-E). Furthermore, a large change in which cells are expressing LAP was observed, in that a minority of LAP+ cells were also positive for the DC marker CD11c in saline and WT treated mice, but in mice treated with Flt3L-MSA, 40-60% of the cells which stain LAP+ also showed CD11c staining (FIG. 3F-G).

T cells isolated from spleens from mice treated with Flt3L-MSA showed an increased percentage of Programmed Cell Death Protein 1 (PD-1) expression, as well as an increased percentage of integrin a487 (a gut-homing integrin) expression relative to both WT and/or saline treated mice. These effects were also observed as trends in the jejunum draining lymph nodes, and the trends became significant as lymph nodes went distally towards the colon, with effects in the ileum draining lymph node being less stark when compared to the effects seen in the colon draining lymph node (FIGS. 3H and I, respectively). Furthermore, Flt3L-MSA showed an increase in Forkhead Box P3 positive (FoxP3+) CD25+ Tregs in all systemic organs, approximately doubling the percentage of CD4 T cells which showed a Treg phenotype in the spleen as well as the intestine draining lymph nodes (FIG. 3J).

### 3. Anti-Drug Antibody Prevention

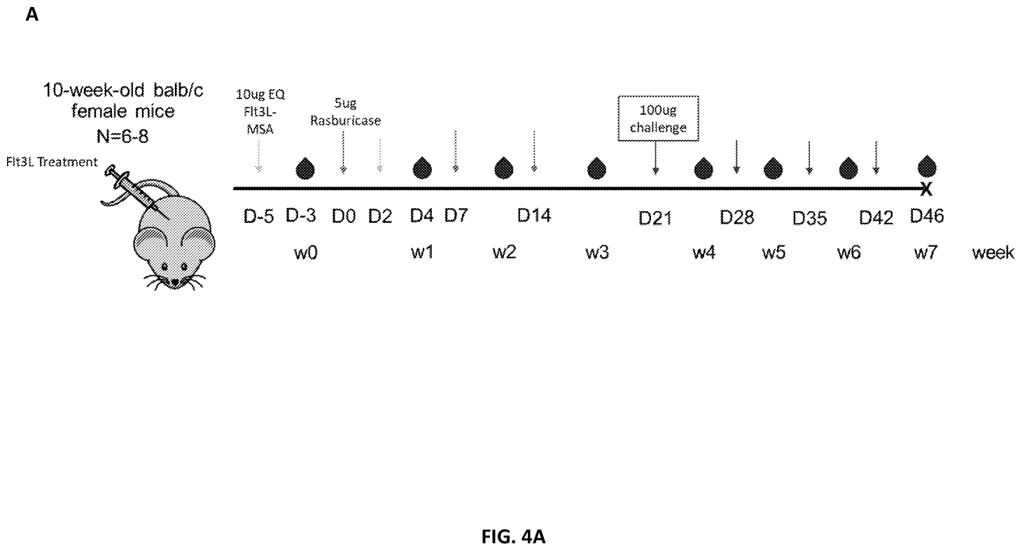

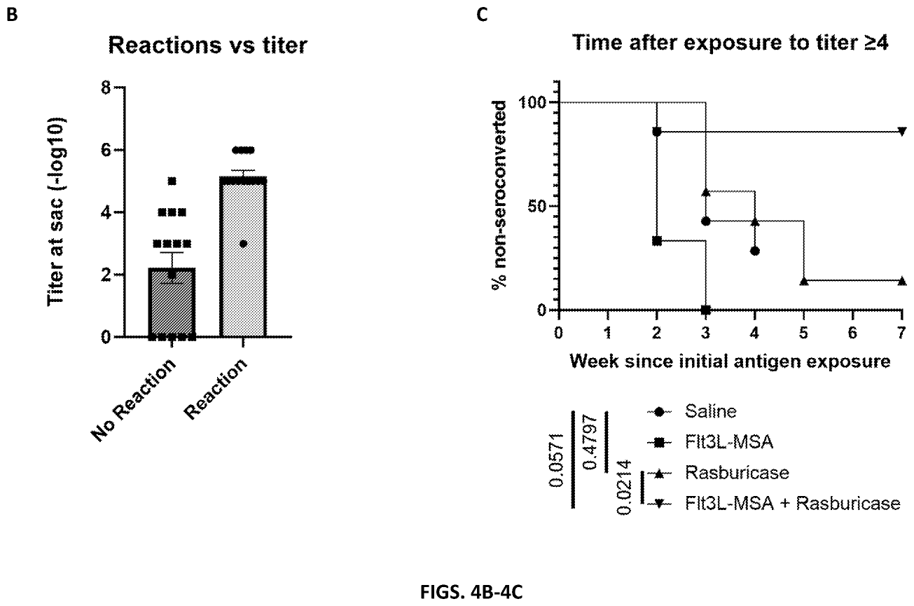

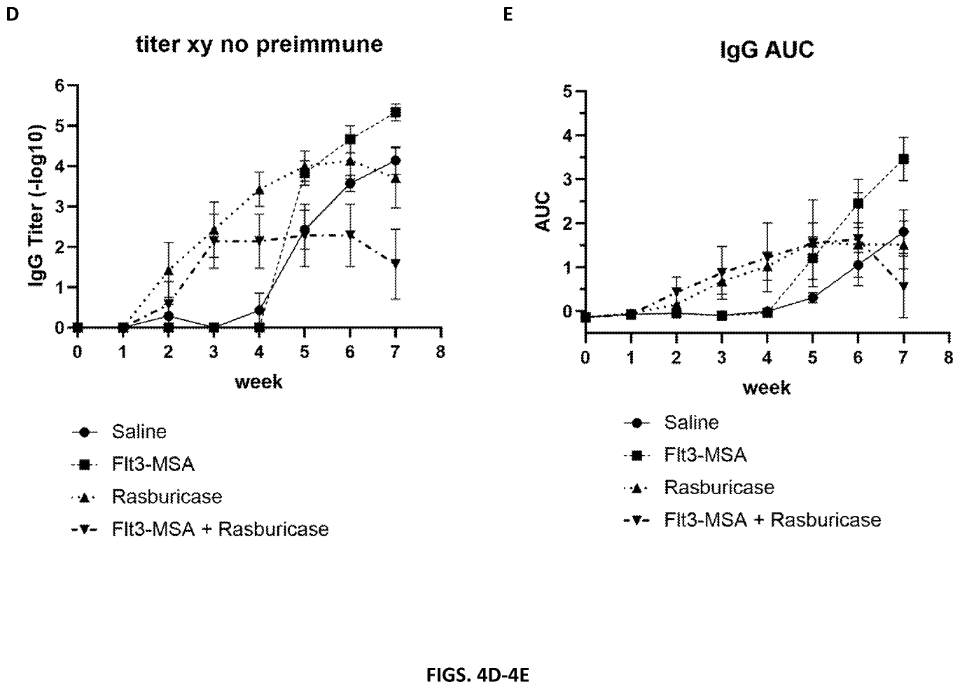

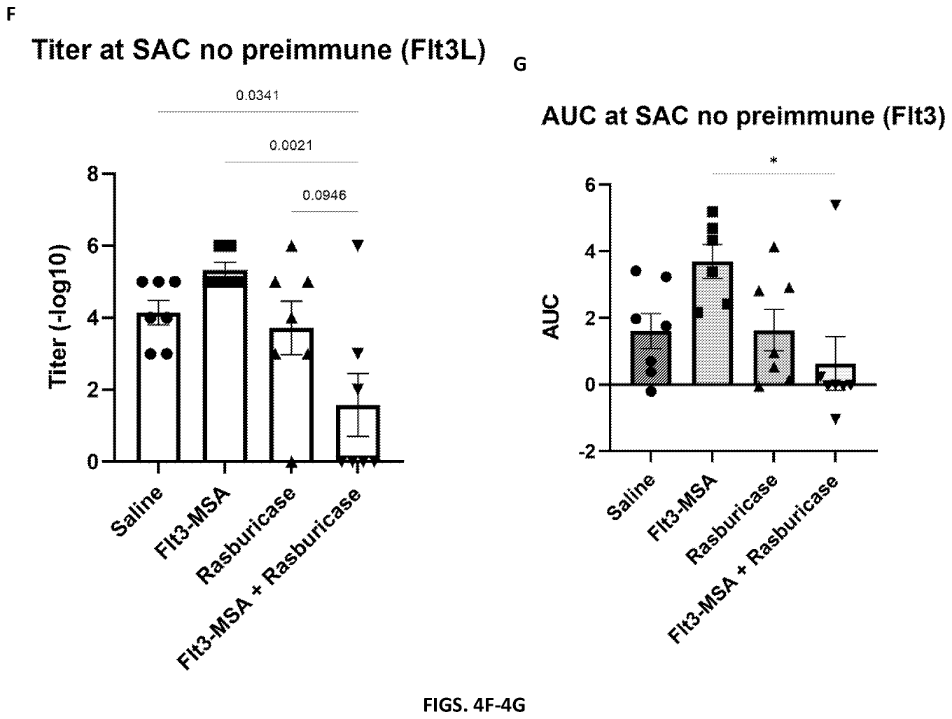

With the rising prevalence of biologic drugs in the healthcare market, antibody mediated reactions to such drugs are also increasing, making these treatments less effective for patients. Recent research shows that Treg induction through tolerogenic DC's is enough to halt antigen specific B cell maturation, and can prevent the antibody response against a drug. As such, the inventors then sought to enhance tolerogenesis to protein therapeutics by combining a low dose of a drug with Flt3L-MSA. As a non-limiting example, this induced tolerance was observed for the very antigenic drug, RASBURICASE™ (FIG. 4A). When examining the total IgG titers of mice which displayed an anaphylactoid reaction (e.g., as evidenced by short gasping breaths as well as a hunched and ruffled posture) upon final challenge, the inventors chose to consider seroconversion as an IgG titer10 of 4 (e.g., as 4/14 mice fell below this level in the no-reaction category while only 1 mouse which reacted fell below this number) (FIG. 4B). When striating on this category, significantly fewer mice treated with a combination of Flt3L-MSA with a low dose of antigen in the “tolerance induction” phase seroconvert were observed when compared to mice treated with low dose antigen alone (FIG. 4C). In addition, near significance to the challenge alone group was observed; however, this effect may be due to the difference in timeline, in that the challenge alone mice were only followed for 4 weeks following antigen exposure while mice treated with low dose antigen were followed for 7 weeks. When analyzing the titers over time, the low dose alone group and the group which received the combination of Flt3L-MSA with low dose antigen showed similar trends in antibody titers through the tolerogenic regime; however, once the mice move into the “challenge” regime, the antibody titers for the group which was only treated with low dose antigen continued to increase over time, while the antibody titers for the combination treatment group remained stable at around the detection limit of the assay (FIG. 4D-E). By the endpoint of the experiment, 100-fold fewer antibodies were observed in the plasma of combination treated mice when compared to challenge only control mice and/or low dose tolerance induction mice. When looking only at titers following the final challenge, a significant decrease (p=0.0341) in antibody titer was observed when comparing Flt3L-MSA with low dose antigen treated mice to challenge only control treated mice. In addition, a near significant trend (p=0.0946) was observed when comparing low dose antigen with and without Flt3L-MSA (FIG. 4F-G).

4. Improving Tolerance Induction by Combining Flt3L-MSA with Oral Immunotherapy.

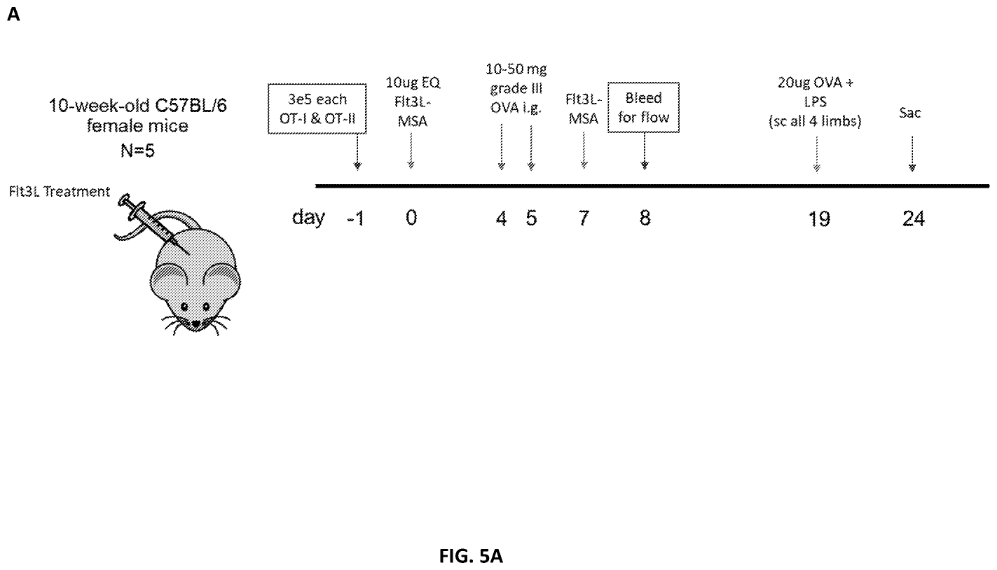

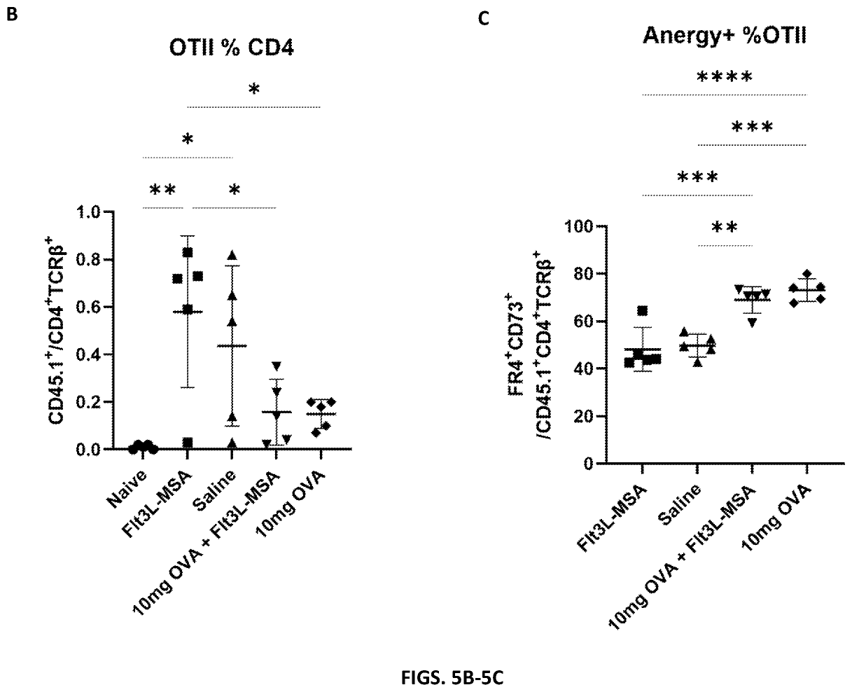

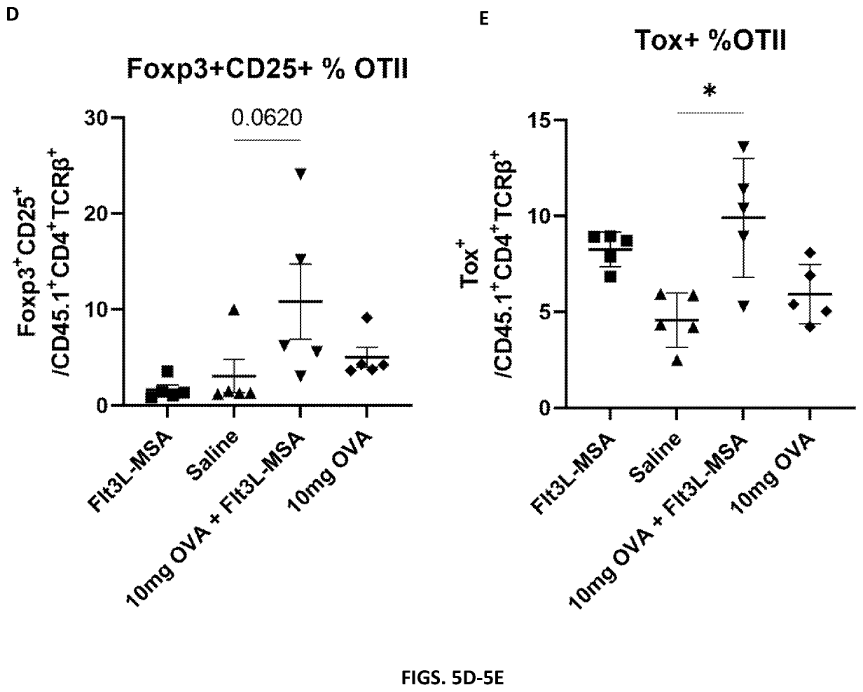

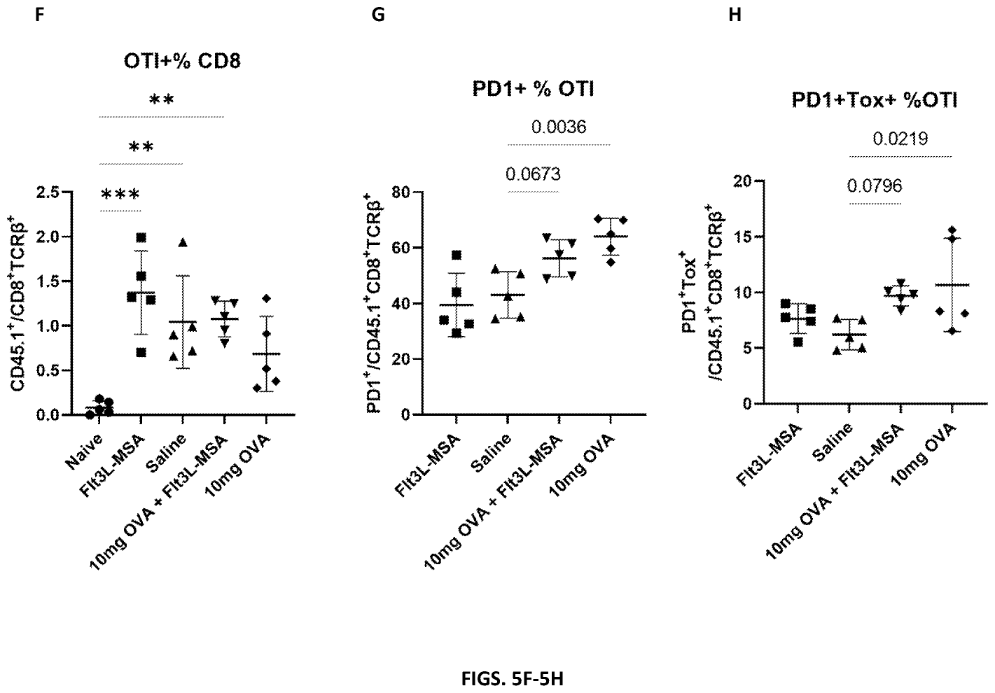

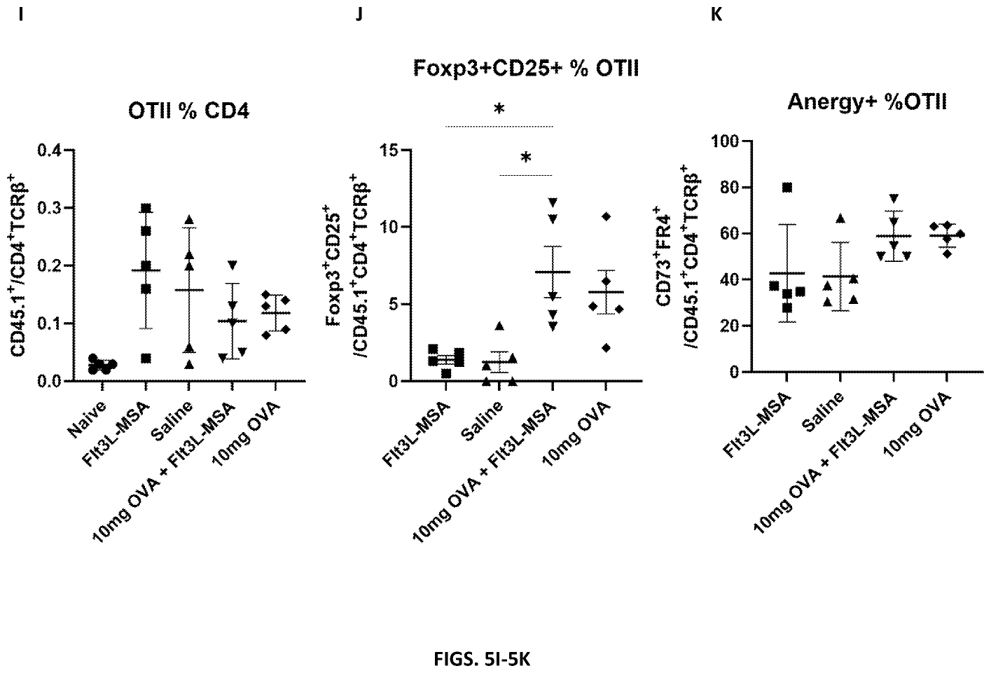

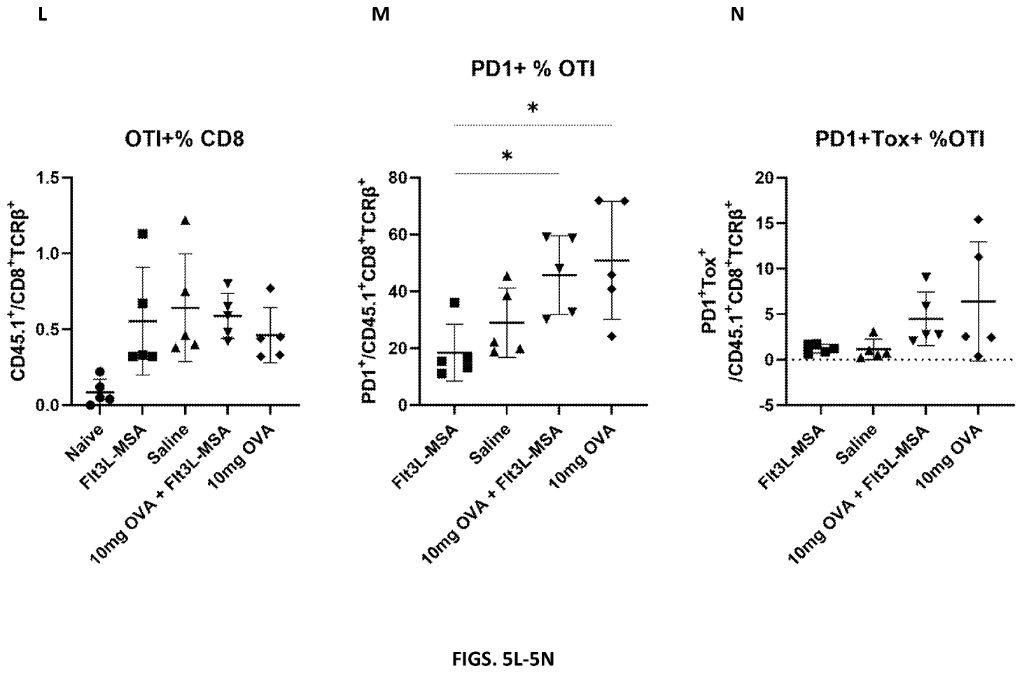

Due to the increase in tolerogenic DC and bulk Treg cells seen in the gut draining lymph nodes (FIG. 3A-J), the inventors next determined whether the addition of Flt3L-MSA to a regimen of oral antigen would benefit oral tolerance induction and prevention of antigen response following challenge. Indeed, mice which received oral antigen showed significantly fewer antigen specific CD4+ T cells in the vaccine draining lymph node. Of the recovered antigen specific cells, a highly significant increase in percent of cells positive for anergy markers Ecto-5′-nucleotidase (e-5NT, also known as CD73) and Folate Receptor 4 (FR4) were observed in mice which received an oral antigen. Additionally, a near significant increase in Antigen specific Tregs was observed, but only in the group treated with both Flt3L-MSA and oral Ovalbumin. Furthermore, a significant increase in antigen specific CD4 T cells expressing the exhaustion transcription factor Thymocyte Selection Associated High Mobility Group Box (TOX) was observed in mice treated with both Flt3L-MSA and an oral antigen (FIG. 5A-E). The antigen specific CD8 compartment in the vaccine draining lymph node was concurrently analyzed. In this compartment, only the mice which received only the oral tolerogen did not show significant increases in antigen specific CD8 T cells following vaccination when compared to naïve mice. However, the results showed significant and near significant trends for increased percentages of cells expressing the exhaustion marker PD-1 and TOX in mice which received oral antigen with or without Flt3L-MSA, respectively (FIG. 5 F-H).

Systemic antigen responses were further investigated through analysis of splenic cellularity after challenge. In the spleen, a significant increase in antigen specific Tregs was observed, but only in the group which received both Flt3L-MSA and oral antigen. Furthermore a non-significant trend towards increased anergy in antigen specific CD4 T cells was observed in both groups which received oral antigen (FIG. 5I-K). Finally, as observed in the challenge draining lymph nodes, there was a significant increase in PD-1 (e.g., a marker of exhaustion) on antigen specific CD8 T cells in both groups which received oral antigen, as well as a non-significant increase in the same cells co-expressing the transcription factor TOX (FIG. 5L-N).

### B. Methods

### 1. Protein Production and Purification

A plasmid containing the murine Flt3L sequence followed by Mouse Serum Albumin (MSA) separated by 2 repeats of a Gly4Ser linker was generated in the pCDNA.3 plasmid backbone under an IgGk leader and excretion sequence with a C terminal 6his tag. The plasmid was transformed into CaCl2 competent DH5a E. coli bacteria and cultured overnight in 2×YT broth. Total plasmid was then purified from culture using a Machery Nagel Maxi prep kit.

Protein production was performed using the HEK293F system. Briefly, cells were cultured in Freestyle 293 media and split to 1e6 cells/mL. On the day of transfection, 25 kDa Polyethylenimine (PEI) was resuspended in OptiPro media to 0.1 mg/mL and the plasmid was separately resuspended in OptiPro media to 1 μg/mL. The PEI mixture was then slowly added to the plasmid mixture to a final concentration of 1:1 and allowed to incubate at RT for 10 minutes. After 10 minutes, the full mixture was slowly added to the HEK cells to a final concentration of 1:25 optipro mixture to cell and culture media. Cells were then incubated at 37° C., 70% humidity, 5% CO2, and orbitally shaken at 140 RPM for 7 days. Following these 7 days, the culture supernatant was centrifuged at 4000 g for 10 minutes prior to 0.22 μm filtration and pH equilibration to a pH of 8 using 1M Tris Base at pH of 9.

The supernatant was then purified using a cytiva Akta avant over a HisTrap HP column at 5 mL/min. After loading, protein was washed using 5 mM Imidazole and eluted using 350 mM Imidazole. Following confirmation of protein presence in the elution peak via SDS PAGE, the fractions were pooled and further purified over a Sephadex 200 μg 16/600 size exclusion column pre-equilibrated in PBS.

### Example 2—Antigen Tolerance Induction Through Use of Flt3L Variants

### A. Results

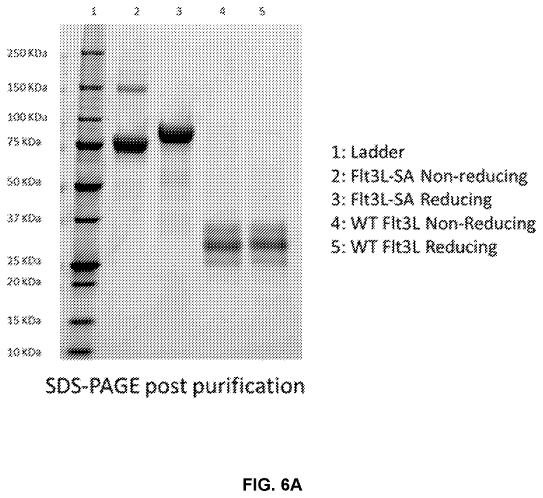

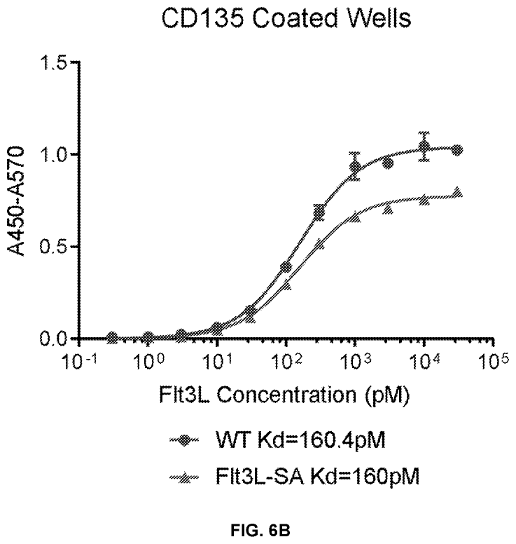

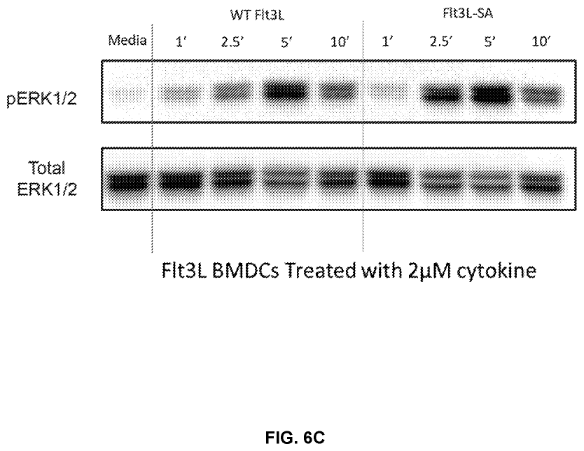

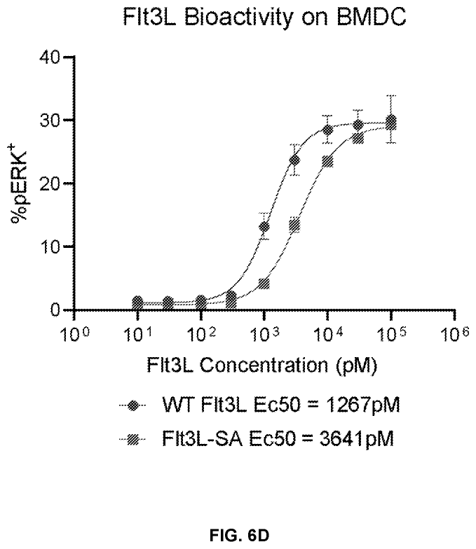

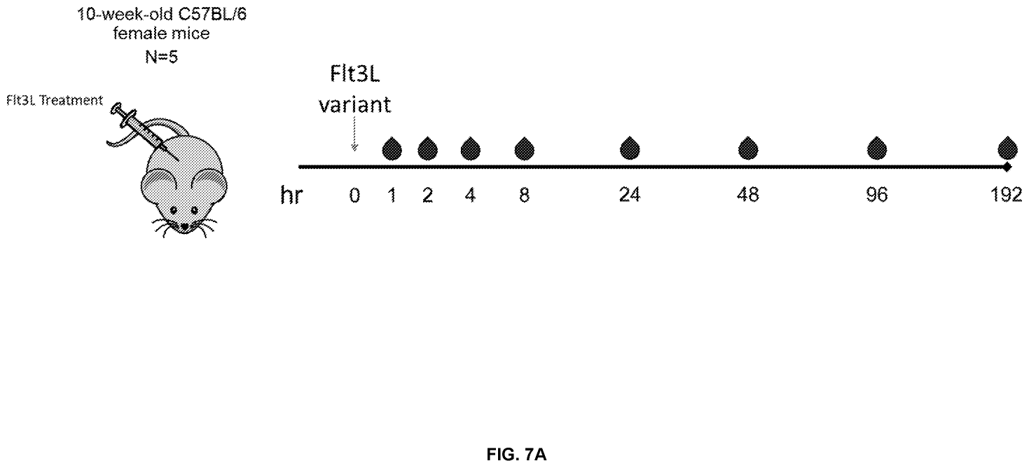

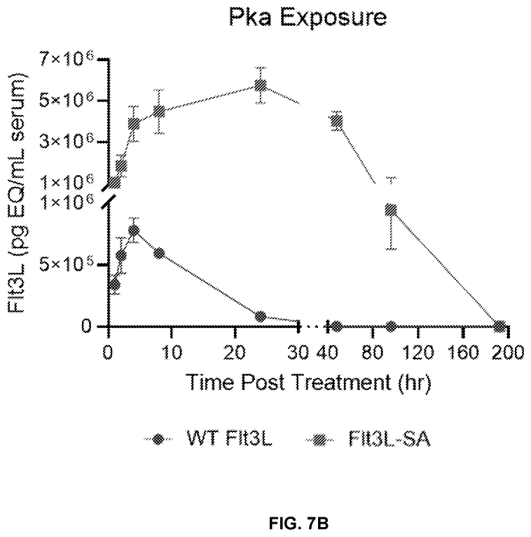

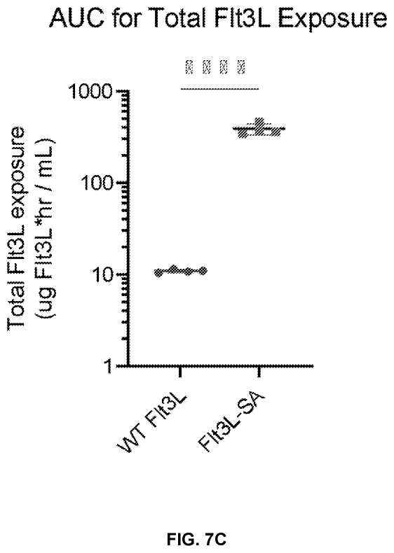

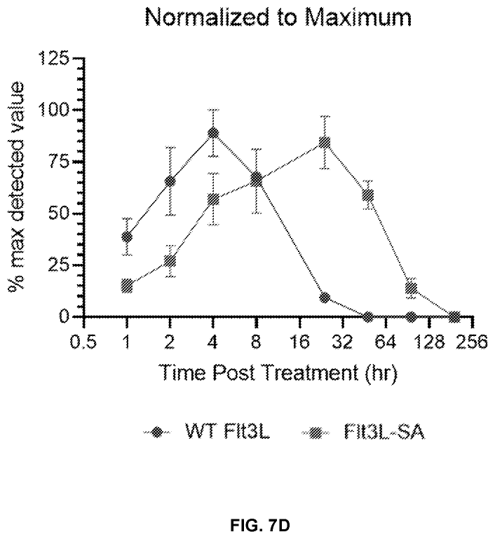

Following production and purification, we see high purity of the two Flt3L constructs (WT Flt3L and Flt3L-SA) via SDS-PAGE (FIG. 6A) as well as confirmed equivalent binding to the receptor CD135 (FIG. 6B). Once binding was confirmed, we next sought to determine bioactivity of the constructs in vitro. Treating starved, Flt3L-generated BMDCs with each construct demonstrated that peak activity, measured by levels of phosphorylated ERK1/2, was seen five minutes after treatment (FIG. 6C). Following a similar procedure as for the western blot based assay, we treated starved Flt3L-generated BMDCs with varying concentrations of the Flt3L proteins and measured ERK1/2 phosphorylation by flow cytometry. In this we see a three-fold difference in relative bioactivity between the two proteins (FIG. 6D). We next sought to check the pharmacological properties of the two constructs. First, we performed a pharmacokinetic study where mice were treated one time s.c. with 10 ug of Flt3L or molar equivalent of Flt3L-SA and bled at indicated time points and Flt3L content in the blood was measured via ELISA (FIG. 7A). In this, we see an extension of time for the SA construct leaving the injection site as well as extending the half-life of the protein. Taking area under this curve, we also see a 30-fold increase in the exposure to Flt3L when fused to albumin (FIG. 7C). Finally, when normalized to the maximum value measured, we see an extension of half life from 10.6 hr for the WT construct to 55.8 hr when fused to albumin (FIG. 7D).

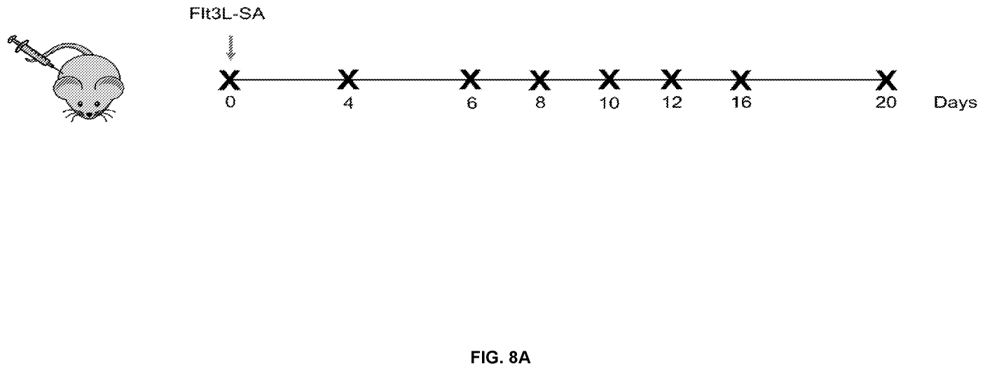

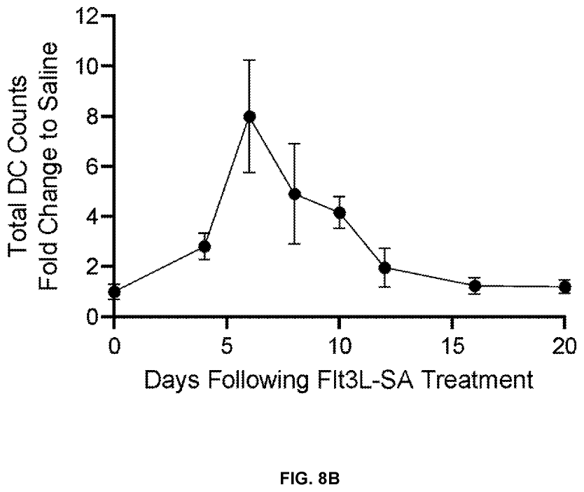

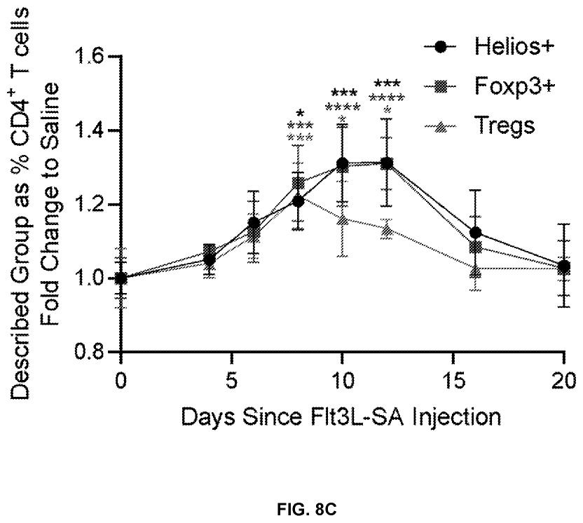

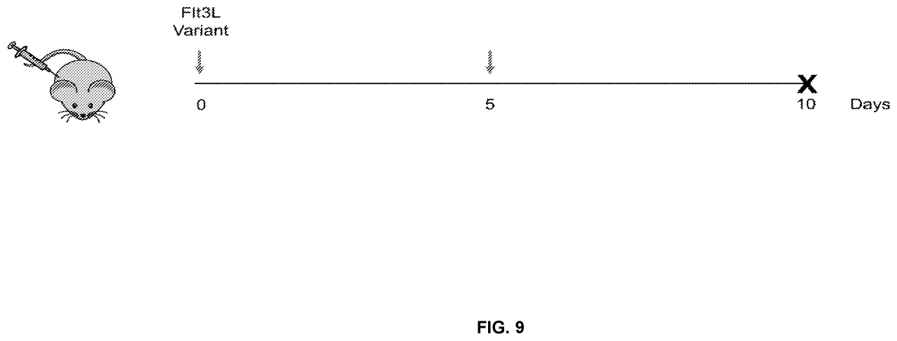

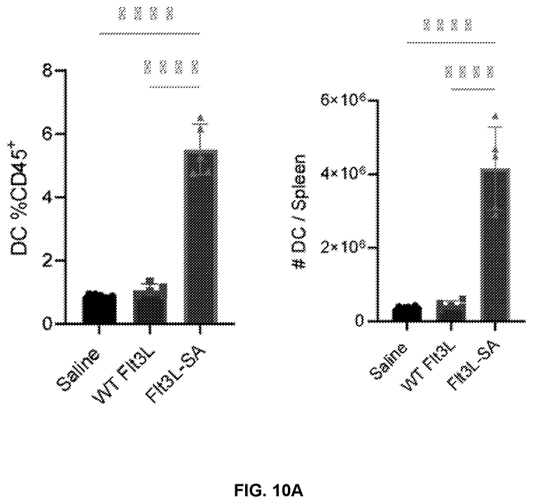

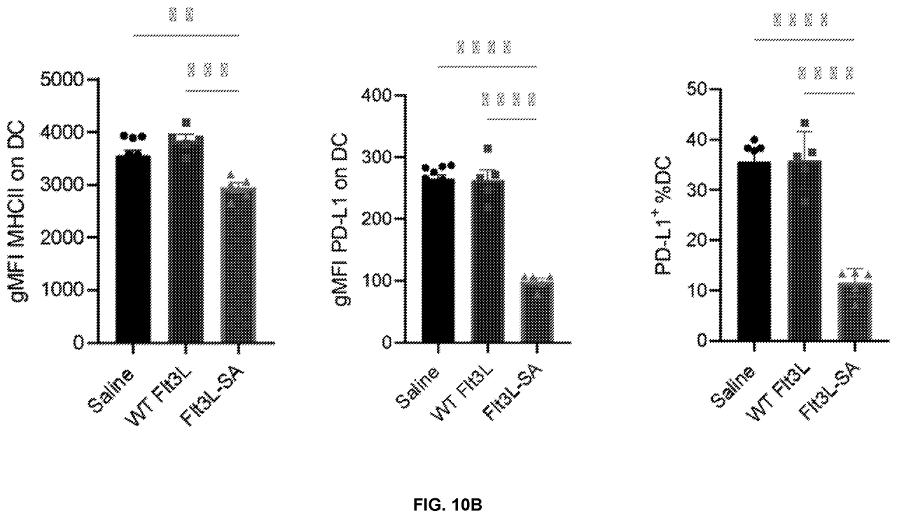

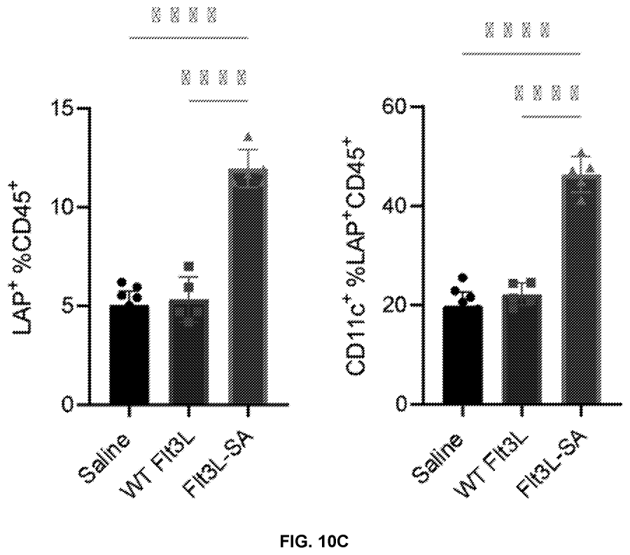

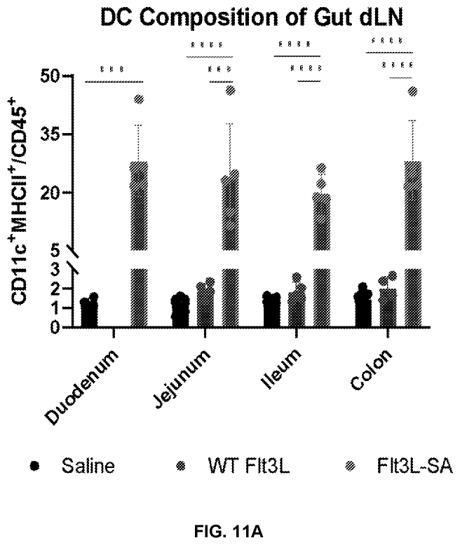

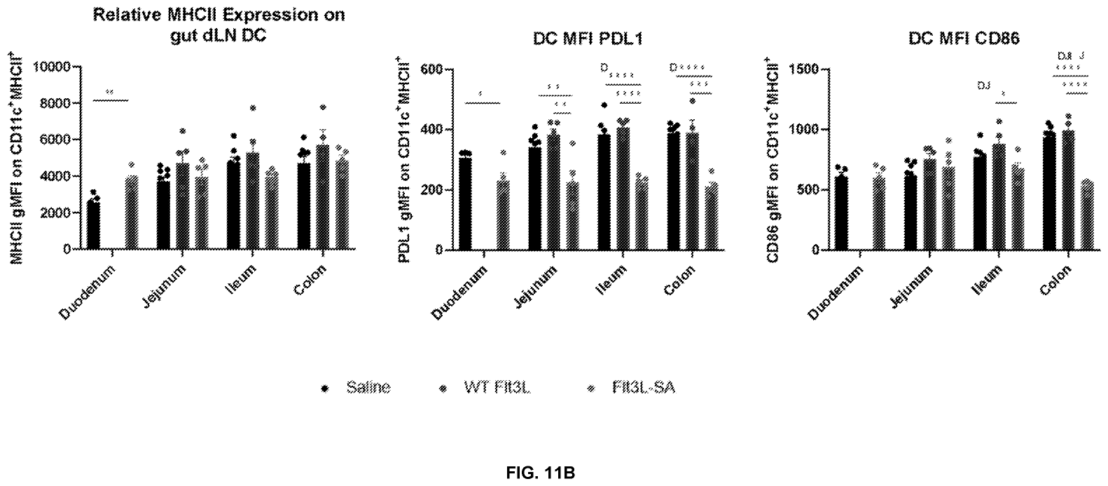

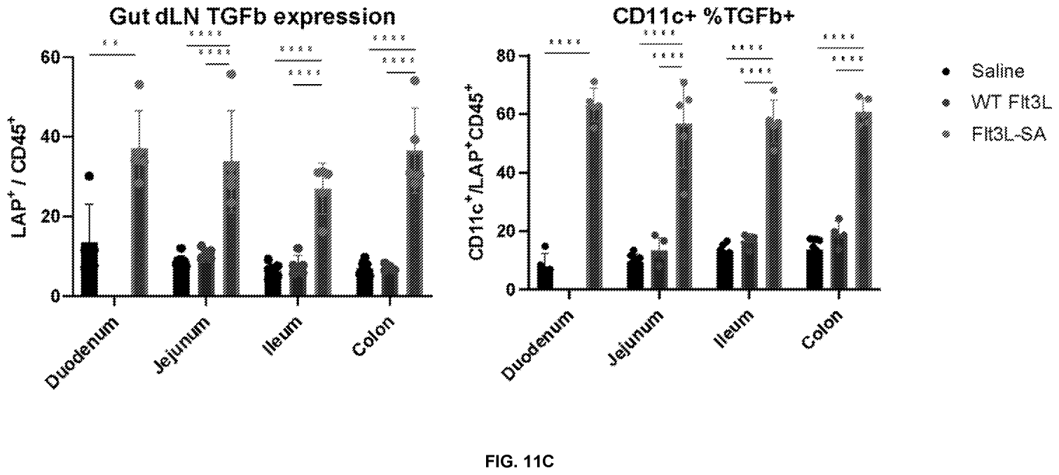

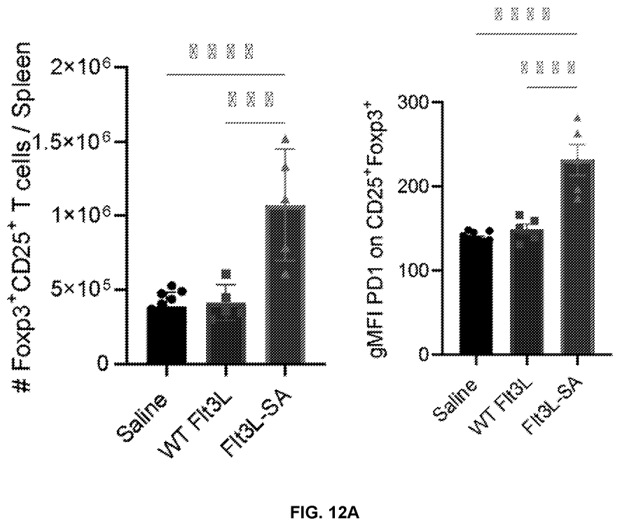

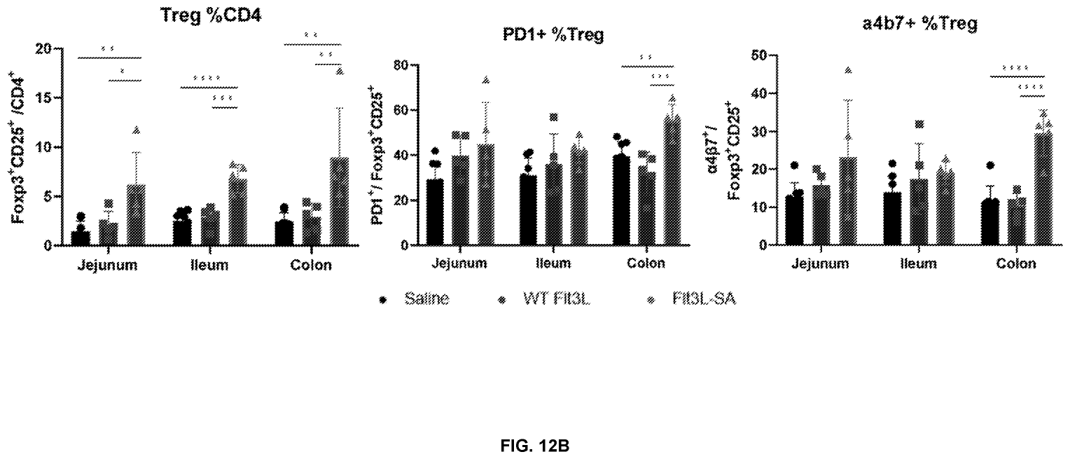

Knowing then that the WT variant is cleared quickly, we next sought to determine the pharmacodynamics of the SA variant. Herein, mice were treated once with 10 ug EQ Flt3L-SA and euthanized at the indicated time points to determine how splenic populations changed after treatment (FIG. 8A). After a single injection, we see an eightfold increase in dendritic cells within the spleen (FIG. 8B) as well as significant increases in the percent of CD4+ T cells staining for regulatory phenotypes via Foxp3, Helios, and Foxp3 with CD25 (FIG. 8C). With pharmacologic information in hand, we next developed a tolerogenic regime using multiple treatments of Flt3L variants and determining how cell populations changed in both the spleen as well as the lymph nodes draining varying areas of the intestines (FIG. 9). We note marked increases in DCs in the spleens of mice treated only with the Flt3L-SA variant (FIG. 10A) and these cells display decreased levels of activation markers (FIG. 10B). Furthermore, in mice treated with Flt3L-SA we see increased numbers of cells expressing the TGF-B precursor, LAP with a majority of these cells staining for the DC integrin CD11c (FIG. 10C). These same effects are also observed in lymph nodes draining intestines with stronger phenotypes than seen in the spleen (FIG. 11A-C). Next characterizing Tregs after treatment, we see three times as many Tregs in the spleens of mice treated with Flt3L-SA and these cells display increased expression of PD-1 (FIG. 12A), potentially suggesting recent antigen experience or proliferation due to antigen recognition in the periphery. Furthermore, we also note significantly increased Tregs in the lymph nodes draining the distal portions of the gut which also seem to demonstrate increased expression of PD-1 and the gut-homing integrin a4b7 especially in the lymph node draining the colon (FIG. 12B).

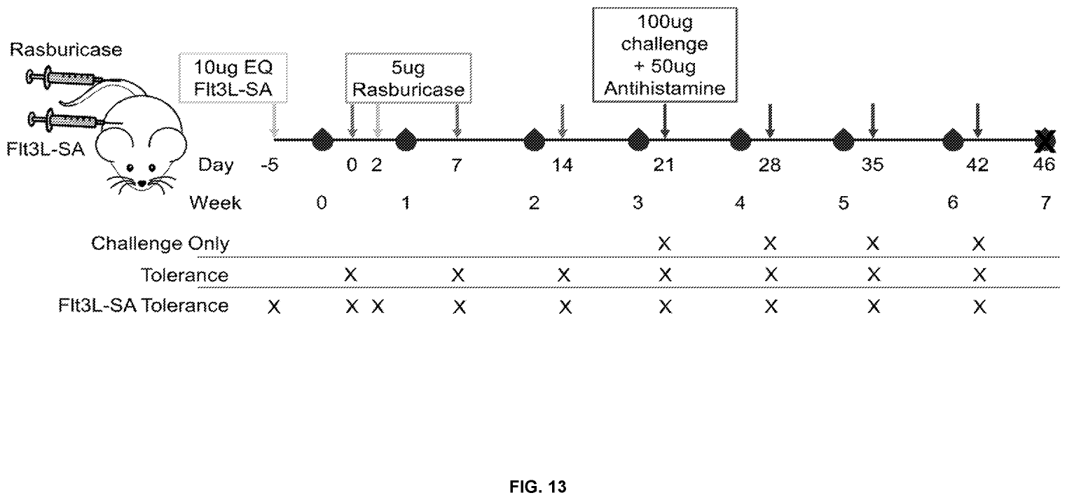

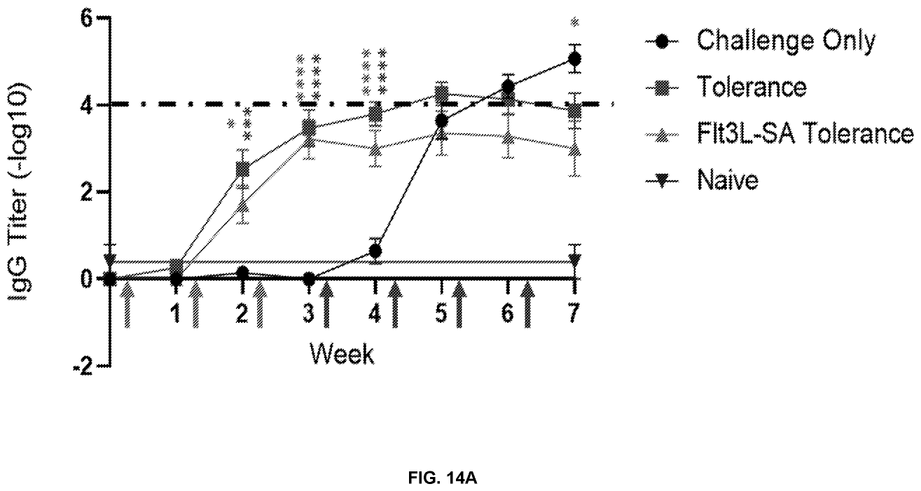

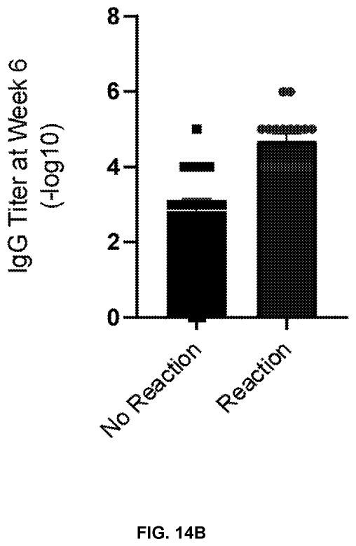

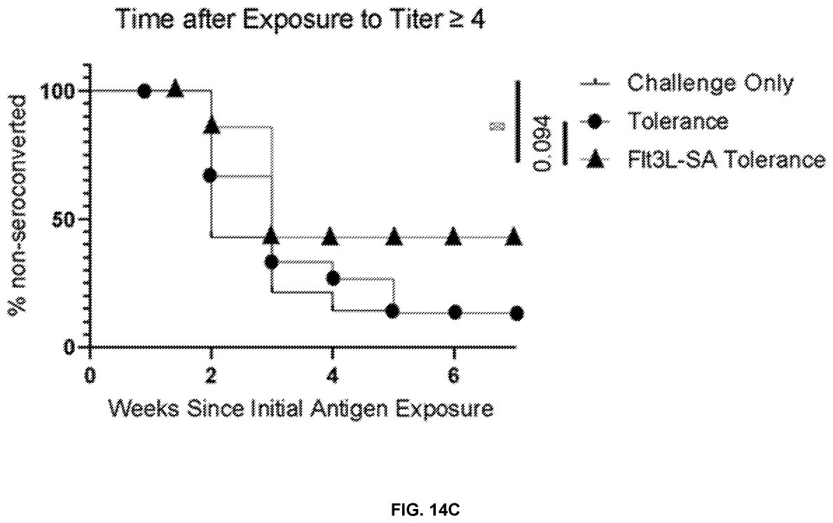

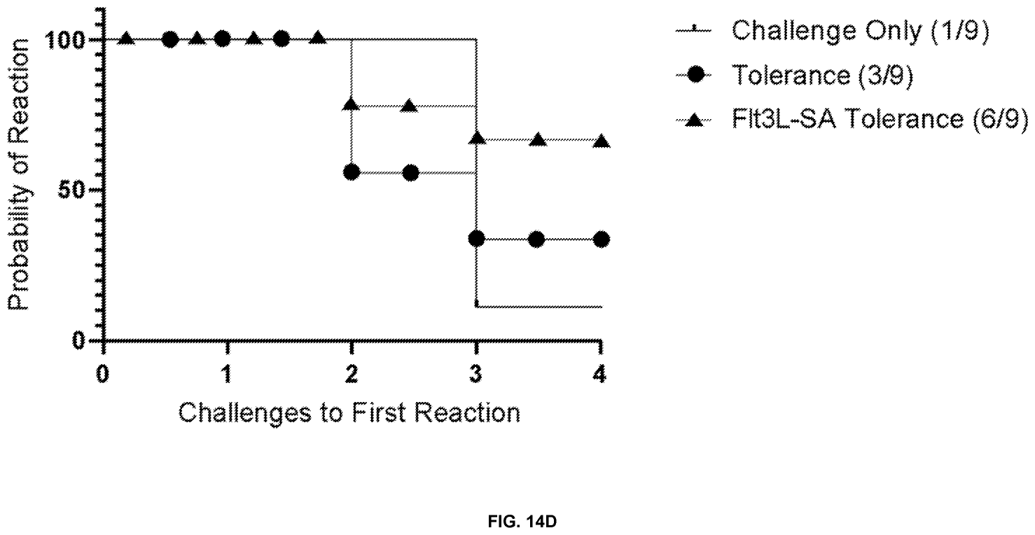

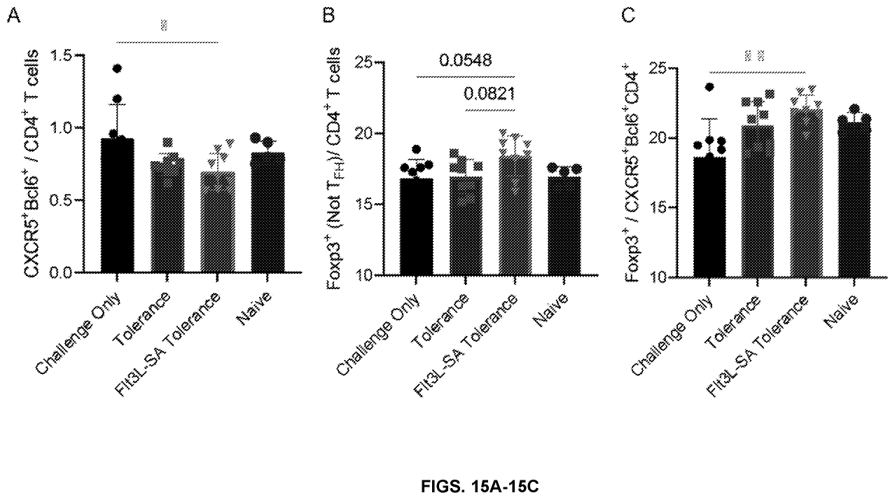

With tolerance regime in hand, we next looked to determine if Flt3L-SA could be used to enhance tolerance induction to foreign proteins in a model of enzyme-replacement therapy (FIG. 13). We note significant reductions in antibodies generated to the enzyme only in the case with the addition of Flt3L-SA to the tolerance regime (FIG. 14A). Furthermore, when tracking which mice generated anaphylaxis-like symptoms at the final challenge dose and comparing that to the antibody level just preceding the dose, we note that an IgG titer of 4 seems to be a cutoff for likelihood of generating these deleterious reactions (FIG. 14B). Using a titer of 4 then as the cutoff for seroconversion to reactions, we note a significant benefit to combining Flt3L-SA in the tolerance regime, where without addition of Flt3L-SA the time to seroconversion tracks similarly to that of no tolerance induction at all (FIG. 14C). We also noted that in tracking when mice begin to show signs of anaphylaxis like reactions after challenges that by adding Flt3L-SA to the regime we see two-thirds of mice show no reaction in comparison to mice treated with just the low-dose antigen in which only one-third are protected (FIG. 14D). Finally, looking at Splenic T cells on day 46 of this experiment, we see a significant reduction in the T follicular helper cells (FIG. 15A), a near significant increase in Tregs (FIG. 15B), and a significant increase in the percent of T follicular helper cells displaying a regulatory phenotype (by expression of Foxp3) (FIG. 15C), all only in mice treated with Flt3L-SA.

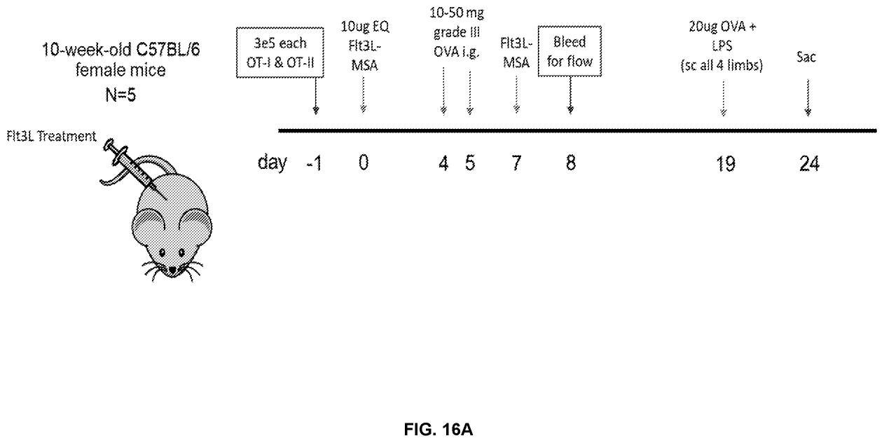

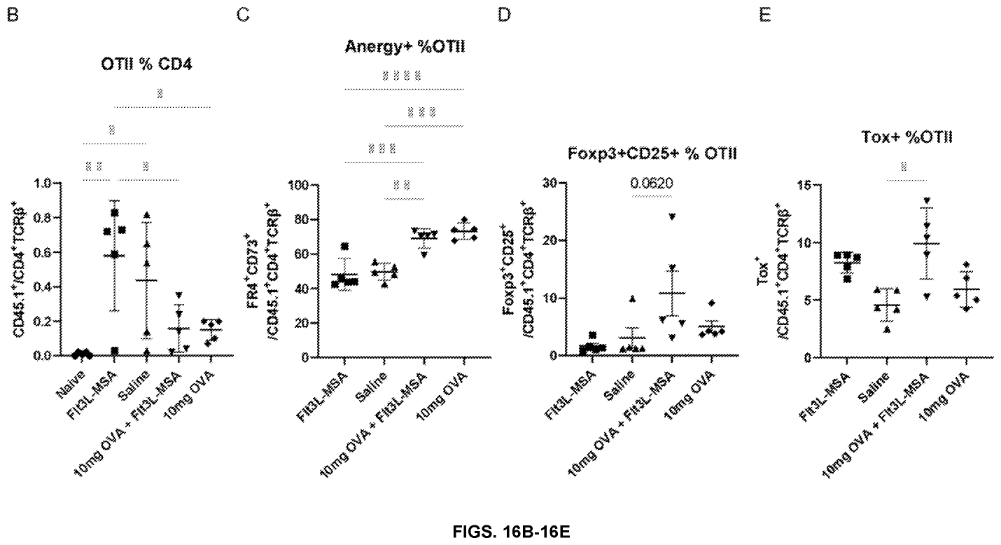

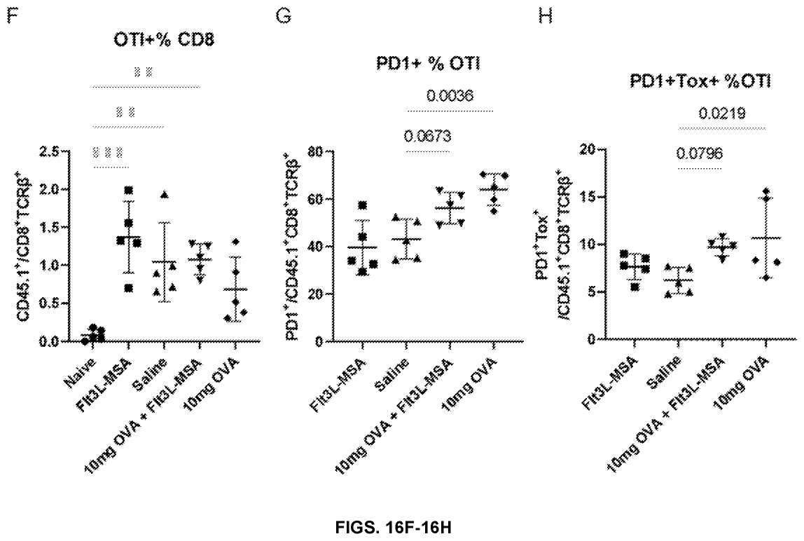

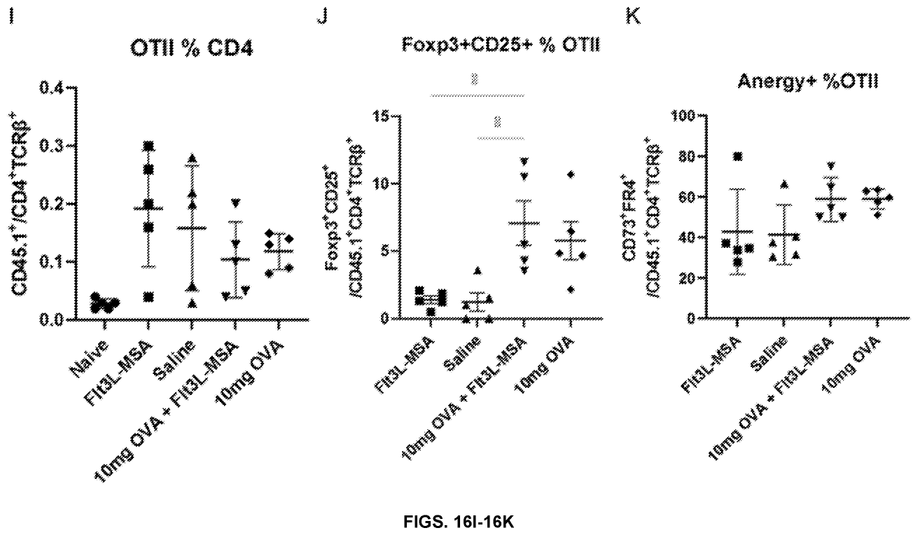

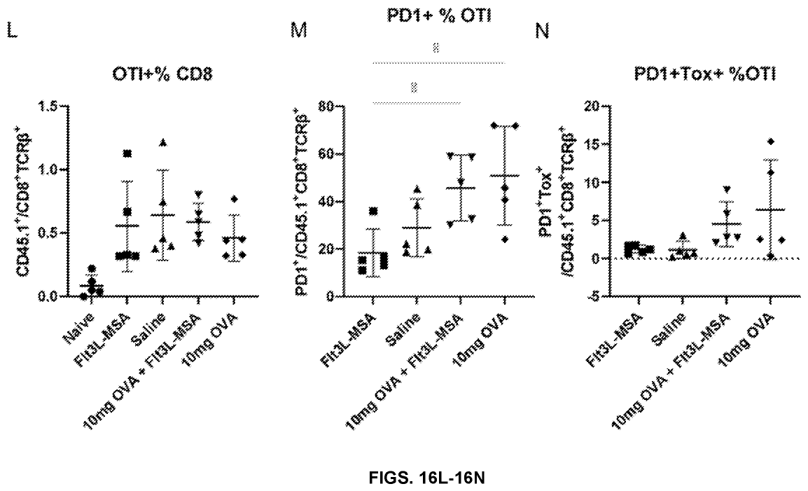

Given the observed effects we see in the gut draining lymph nodes, we also sought to determine if addition of Flt3L-SA could modify how the mice react to oral antigen exposure and if that could then be transitioned to protect against peripheral challenges (FIG. 16A). Looking at proliferation of antigen specific T cells in the vaccine draining LN, we see decreased percentages of antigen reactive CD4 T cells in mice given oral antigen as well as increased markers of T cell anergy (FIG. 16B-C). However, of these antigen specific T cells, we see a near significant increase in Tregs in mice receiving both oral antigen and Flt3L-SA (FIG. 16D) as well as Flt3L-SA inducing the T cell marker of exhaustion TOX (FIG. 16E). Next looking at antigen specific CD8 T cells in the vaccine dLN, we see a marked decrease in responding cells in mice treated with only oral antigen (FIG. 16F), but of the cells present, there in also an increase in expression of markers of exhaustion (FIG. 16G-H). Finally, we looked at systemic reactions (in the spleen) following vaccination. Systemically, we note that effects are primarily driven by the addition of oral antigen with decreased antigen specific T cells, increased Tregs, and increased expression of anergy markers on CD4 T cells (FIG. 16I-K). Furthermore we see no meaningful differences in systemic antigen-specific CD8 T cells nor in expression of markers of exhaustion (FIG. 16L-N).

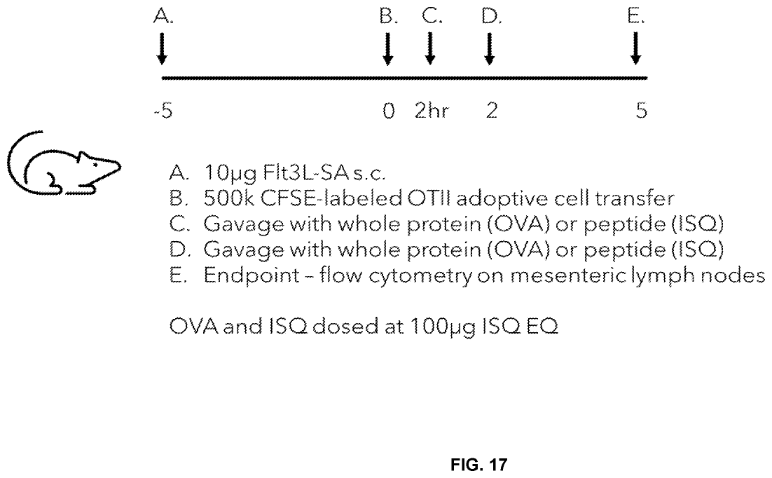

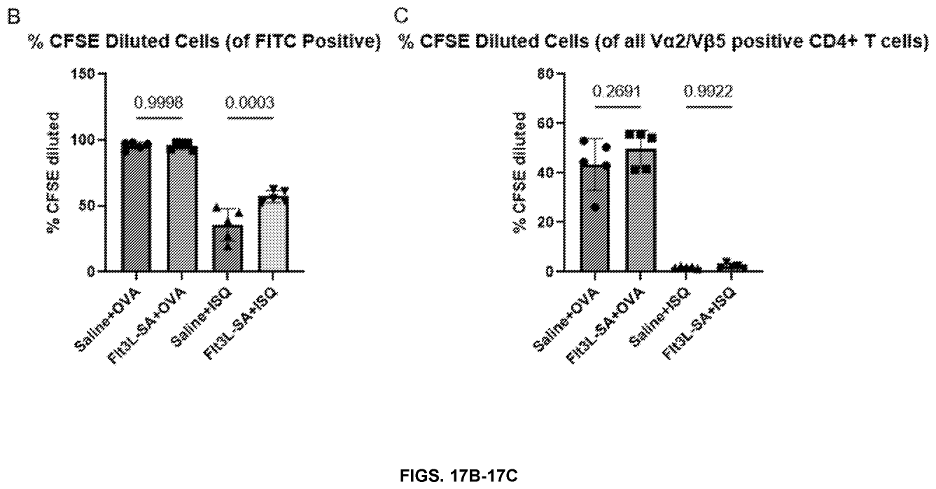

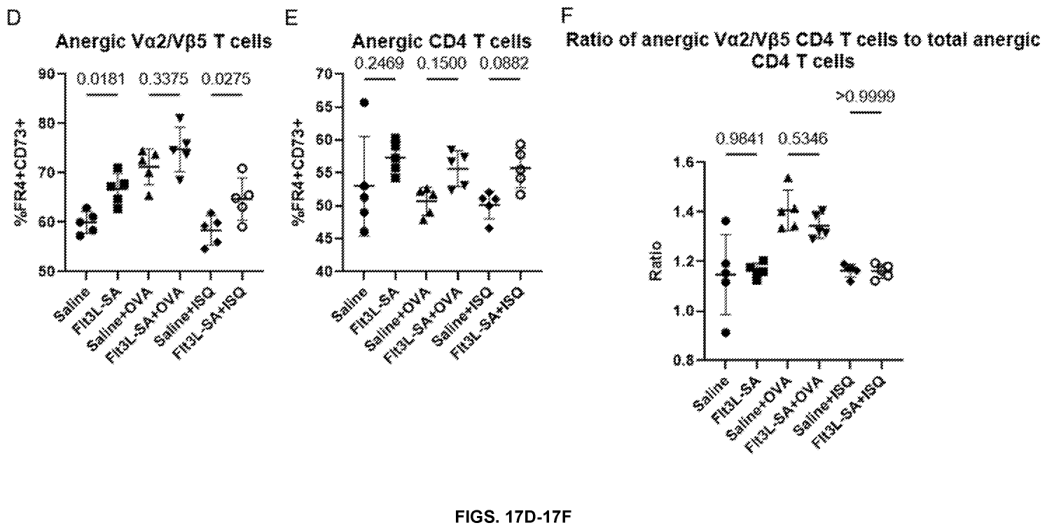

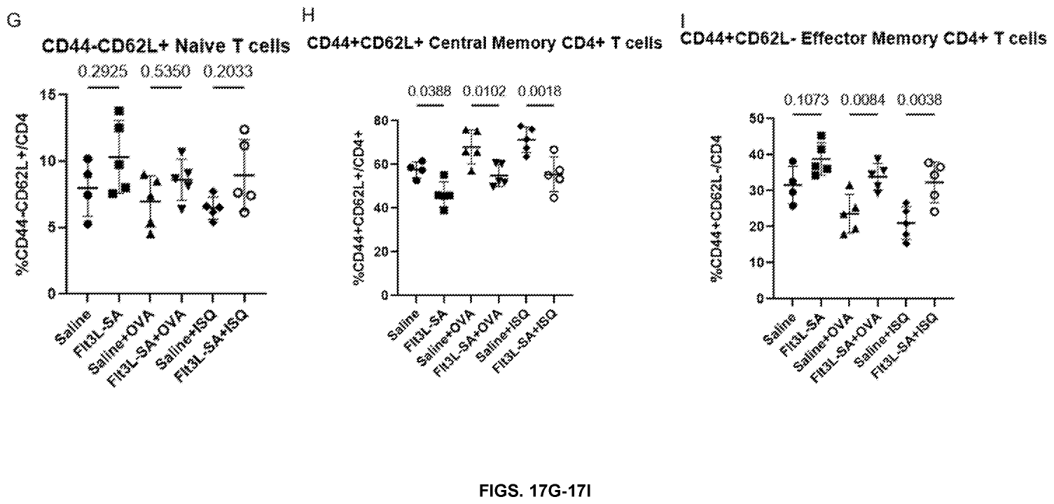

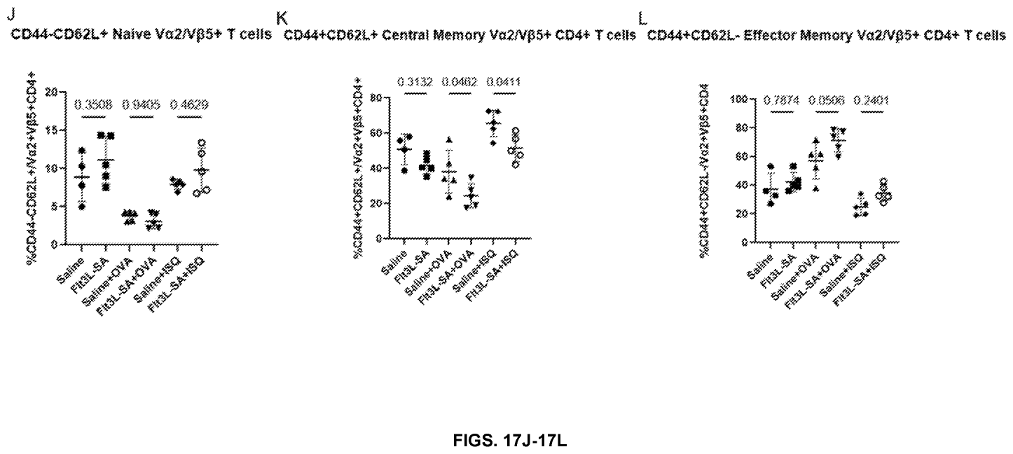

Five days after Flt3L-SA administration, an adoptive transfer of 500,000 CFSE-labeled OTII cells was performed, followed by two i.g. administrations of OVA or the MHC class II-binding peptide OVA 323-339 (ISQ) at 2 hours and 2 days post-transfer. Mice were euthanized five days following the adoptive transfer for flow cytometric analysis of their mesenteric lymph nodes (FIG. 17A).

Within live CD4+ T cells, those expressing the Vα2/Vβ5 TCR were focused on to further isolate the OTII population, known to use this TCR (though some of the endogenous repertoire will as well). Within the FITC+ in this population (presumed to be CFSE stained to some extent), adding Flt3-SA to ISQ gavage led to a significant increase in CFSE dilution among the OTIIs (FIG. 17B). A similar trend was observed when the percentage of CFSE diluted cells was looked at as a percentage of total Vα2/Vβ5 CD4+ T cells for OVA, but this did not reach significance (FIG. 17C). Flt3L-SA treatment was able to promote T cell anergy (as indicated by FR4+CD73+ populations) in both the bulk and Vα2/Vβ5 CD4 T cell populations, although percentages were substantially higher in the Vα2/Vβ5 CD4 compartment. This was despite the OTIIs transferred into the mice only being transferred 5 days post Flt3L-SA administration (FIG. 17D-E). The ratio of anergic cells in the Vα2/Vβ5 and bulk compartments within each treatment showed no significant differences within paired treatment groups, suggesting that the compartments were influenced about equally by the Flt3L-SA administration (FIG. 17F).

We observe that within the mesenteric lymph nodes as well, Flt3L-SA treatment results in no evidence of change to the naive CD4 cell compartment (FIG. 17G), but there is a downregulation in central memory CD4 T cell compartment (FIG. 17H) and corresponding upregulation in the effector memory CD4+ T cell compartment (FIG. 171). Within the Vα2/Vβ5 CD4+ T cell compartment, there is similarly no change in the naive CD4 T cell compartments (FIG. 17J), and downregulation in the central memory CD4 T cell compartment (FIG. 17K), however increases in the effector memory compartment within this population failed to reach significance (FIG. 17L).

### B. Methods

### 1. Animals:

C57BL/6 mice were purchased from Jackson Labs and housed at the University of Chicago Animal Facility. All procedures were approved by the University of Chicago Institutional Animal Care and Use Committee and performed on protocol #72449. Injections were diluted to the appropriate concentration in 50 uL sterile saline for S.C. injections or 100 uL for I.V. or I.P. treatments. Mice were euthanized via CO2 asphyxiation as approved by UChicago ARC.

### 2. Protein Production and Purification:

Plasmids encoding Flt3L or Flt3L-SA were diluted into OptiPro transfection media to a concentration of 50 ug/mL. Linear, 25 kDa polyethylemine was then also diluted to a concentration of 100 ug/mL before slowly adding the PEI mixture to the DNA to a final ratio of 1:1 (v: v). This mixture was incubated at room temperature for 10 minutes without agitation to allow DNA/PEI complexation. After complexation, 1 mL of transfection reagent was slowly added with agitation to 25 mL of HEK293F cells grown to a concentration of 1e6 cells/mL. Transfected cells were then incubated at 37 C for one week with constant shaking before protein harvesting. At harvest, cells were removed by centrifugation at 4000G for 10′ before filtering the supernatant through 0.22 μm filter. Filtered supernatant was then pH adjusted to about pH of 8 using 1M Tris at a pH of 9. This supernatant was then loaded over a HisTrap HP (Cytiva) column on a GE AKTA Avant FPLC. Once loaded, the column was then washed using 50 mM imidazole to remove loosely bound proteins and then the protein was eluted with 400 mM imidazole. Protein eluted was further purified over a superdex 200 pg column (Cytiva) in PBS, pooling only pure fractions of the correct size. The final protein was then tested for LPS using a HEK TLR4 cell line with quantiblue to confirm endotoxin free samples before aliquoting and freezing.

### 3. SDS-Page:

Protein was prepared according to buffer instructions for reducing and non-reducing preparations before boiling for 15 minutes. 10 ug of protein in sample buffer was then added to each lane of a stain-free gel (Biorad 4568096) before running until the dye front reached the bottom using TGS buffer. Gel activation and imaging occurred on a ChemiDoc XRS+.

### 4. CD135 Binding ELISA:

High-bind plates (Corning #9018) were coated by incubating overnight at 4 C with 10 nM recombinant CD135 (R&D 768-F3-050) in 50 nM sodium bicarbonate buffer at pH 9.2. Following coating, plates were washed in PBST before blocking in 1× reagent diluent (R&D DY995) for 2 hours at RT. Samples were then diluted to the indicated concentration in triplicate using the same reagent diluent. Blocked wells were washed and then sample was added and incubated for one hour. Sample was removed before addition of the detection antibody (R&D 841481) at the recommended concentration and incubated for 1 hour followed by washing and addition of Streptavidin-HRP (R&D 890803) at the indicated concentration for a 30′ incubation. Wells were then washed before the addition of TMB (Millipore ES-001). TMB was incubated while protected from light until saturation was reached, at which point the reaction was stopped using 10% Sulfuric Acid. Quantification occurred by reading the absorbance at 450 nm and 570 nm.

### 5. BMDC Generation:

BMDC were generated according to a modified Lutz protocol. Briefly, bone marrow was flushed from the long bones of healthy 6-15 week-old C57BL/6 mice into RPMI 1640 media and filtered over 100 um filters. On day 0, 3 million nucleated cells were plated in 10 mL of a modified Lutz media (RPMI 1640 supplemented with 10% FBS, 1% Pen/Strep/L-glutamine, 50 uM beta-mercaptoethanol, 25 mM HEPES, 20 ng/mL GM-CSF, 200 ng/mL Flt3L) in 100 mm non-tissue culture treated petri dishes. Cells were fed by the addition of 10 mL of the initial media (with GM-CSF and Flt3L) on day 3. On day 6, media was refreshed by removing 10 mL and centrifuging the cells before resuspension in 10 mL of fresh, complete media (GM-CSF and Flt3L). Non-adherent cells were harvested on day 9 for use and starved for 2 hours in incomplete RPMI 1640.

### 6. Phospho-ERK1/2 Western Blot:

1 million starved BMDCs were resuspended into 100 μL of incomplete RPMI. 100 μL of 4 uM Flt3L or Flt3L-SA in incomplete RPMI was then added to each tube for the indicated time, at the end of which 1 mL of ice-cold PBS was added and cells were placed on ice before centrifugation at 4 C. Supernatant was then removed and cells were lysed using RIPA buffer (Thermo 89901) with protease and phosphatase inhibitor added (Thermo A32959, 1 tablet/10 mL RIPA). Cells were incubated in the RIPA buffer for 5 minutes on ice before centrifugation at 17,000 G for 10 minutes. Supernatant was taken and total protein quantified via BCA (Thermo 23227). Cell lysate was prepared in fresh reducing running buffer and run on SDS-PAGE as previously described. Protein was transferred from the gel to a PVDF membrane using a wet transfer system before blocking the membrane with 5% BSA solution in TBST. Membrane was probed using 1:5000 dilution of anti-pERK1/2 (Biolegend 369502) for 2 hours followed by HRP-conjugated anti-mouse (CST 7076S). Detection occurred using Clarity substrate (Biorad 1705060) before stripping the membrane with Restore Stripping Buffer (Thermo 21059). The procedure was then repeated using 1:5000 dilution of anti-total ERK (Biolegend 686902) and HRP conjugated Anti-Rat (Jackson Immuno 112-036-003) at 1:10,000 to probe for total ERK1/2.

### 7. Phospho-Flow for Phospho-ERK1/2:

Starved BMDCs were plated in a 96 well plate at 500,000 cells in 100 μL of incomplete RPMI. Warmed cytokine solution was then added to each well as a 2× concentration in 100 μL of incomplete RPMI before incubation for exactly 5 minutes. At the end of the incubation period, 50 μL of warmed 5× Lyse/Fix buffer (BD 558049) was added to each well before incubation at 37 C for 10 minutes. Cells were then washed with PBS before resuspension in ice cold Perm Buffer (BD 558050) and a 15 minute incubation on ice. Cells were then washed twice in FACS buffer (PBS+2% FBS+1 mM EDTA) before staining with anti-pERK (as described in methods for flow cytometry staining) and acquisition on a BD Fortessa.

### 8. Pharmacokinetic Study:

Mice were treated one time via S.C. injection and blood was collected into Lithium-Heparin coated tubes at the indicated time points following treatment and plasma isolated following centrifugation at 10,000G for 10′ before freezing. Once all timepoints had been collected, Flt3L content in the plasma was quantified via ELISA (R&D Dy427) using Flt3L standard for mice treated with WT Flt3L and using an equimolar Flt3L-SA standard for Flt3L-SA treated mice. Plasma was diluted 100-10,000× and the lowest dilution for each timepoint which did not oversaturate the standard was taken and converted before accounting for dilution.

9. Preparation of Single-Cell Suspensions from Whole Organs:

After euthanasia, spleens and lymph nodes were harvested into 0.5 mL of complete DMEM and placed on ice. Once all tissues from all mice were removed, spleens were pushed through a 70 um filter and washed with incomplete DMEM. Suspensions were then centrifuged at 1750 RPM for 7 minutes before resuspension in 3 mL of ACK lysis buffer (Thermo 1049201) followed by a 5′ incubation before dilution in incomplete DMEM. Cells were then centrifuged as previously stated before counting and a final resuspension in complete DMEM at a concentration of 20 million cells/mL. Lymph nodes were processed by the addition of Collagenases D and IV at a final concentration of 1 mg/mL each enzyme (Roche 11088866001 and Worthington LS004188, respectively). Lymph nodes were incubated at 37 C for 45′ before processing in a manner similar to spleens without the ACK lysis step.

### 10. Inhibition of Protein Transit for LAP Staining:

Conditions where LAP staining was performed had a pre-incubation step in GolgiPlug (BD 555029) and GolgiStop (BD 554724) to increase sensitivity of the staining. Briefly, 2 million isolated cells were plated in Complete RPMI (RPMI, 10% FBS, 1% P/S) with 1× each inhibitor. Cells were then incubated at 37 C for 4 hours before proceeding with flow staining, as outlined below, using the CytoFix/CytoPerm kit for intracellular staining.

### 11. Blood Collection:

Blood was collected into Lithium-heparin coated tubes via submandibular cheek bleed. Experiments requiring staining of blood cells were then spun at 300G for 10 minutes before ACK lysis and flow staining. To remove plasma alone, tubes were spun at 10,000G for 10 minutes before pipetting off the plasma and freezing in PCR tubes.

### 12. Determination of Antigen Specific IgG Titers:

Corning ELISA plates were coated with antigen at 10 ug/mL of protein in 50 mM Carbonate buffer at a pH of 9.6 overnight before blocking the next morning using 1×Casein buffer (Sigma B6429-500ML) for 2 hours. Samples were diluted from a titer of 2-9 in the same blocking buffer and applied to the wells before incubation for 2 hours at RT. Following washing sample from the wells, HRP-conjugated anti-mouse IgG (Southern Biotech) was then added to the well and incubated at RT for 1 hr. Antigen specific IgG was then detected by incubating with TMB (Millipore) for 18 minutes at RT before the addition of stop solution (3% H2SO4+1% HCl). OD at wavelengths of 450 and 570 nm was then measured on an Epoch Miroplate Spectrophotometer (BioTek). Titer was determined as the log-transformed dilution at which the background subtracted absorbance was greater than 0.01.

### 13. OT Isolation and Adoptive Transfer:

Spleens were removed from transgenic mice expressing the OT-I or OT-II receptor and taken to single cell suspension as described previously. CD4 or CD8 T cells were then isolated using STEMCell kits (#19852 and #19853) before washing and resuspension in plain DMEM. Cells were then injected IV at the indicated times.

### 14. Oral Antigen Delivery:

Grade III Ovalbumin was resuspended in PBS at relevant concentrations before sterile filtration through a 0.22 μm filter. Mice were then gavaged with 200 μL of the antigen while awake.

### 15. Staining for Flow Cytometry:

2 million isolated cells were plated per sample before washing with plain PBS. Viability stain was diluted in plain PBS at 1:500 with the addition of Fc Block before adding 50 μL/well and incubation on ice for 15′. Viability dye was quenched by washing with FACS buffer (PBS+2% FBS+1 mM EDTA) before addition of surface staining antibodies in a 1:1 dilution of Brilliant Stain Buffer (BD 563794) in FACS buffer. Surface staining occurred for 20′ at RT before washing in plain PBS and subsequent fixation. Samples not requiring intracellular staining were fixed for 20′ on ice using 2% PFA in PBS. Samples requiring only intracellular cytokine staining were fixed for 20′ on ice using the BD Cytofix/Cytoperm kit (BD 554714) before washing and intracellular staining for 1 hr-O/N at 4 C. Samples staining for nuclear factors (with or without cytokine staining) were fixed using the Foxp3 Transcription factor staining set (Thermo 00-5523-00) for 45-60′ on ice before intracellular staining for 1 hr-O/N at 4 C. After fixation and intracellular staining, cells were washed and resuspended in FACS buffer for data acquisition on a 5 laser BD fortessa.

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

### REFERENCES

The following references, to the extent that they provide exemplary procedural or other details supplementary to those set forth herein, are specifically incorporated herein by reference.
1. B. G. de la Torre, F. Albericio, The pharmaceutical industry in 2020. An analysis of fda drug approvals from the perspective of molecules, Molecules 26, 1-14 (2021).
2. T. T. Hansel, H. Kropshofer, T. Singer, J. A. Mitchell, A. J. T. George, The safety and side effects of monoclonal antibodies, Nat. Rev. Drug Discov. 9, 325-338 (2010).
3. L. Mayer, Y. Young, Infusion reactions and their management, Gastroenterol. Clin. North Am. 35, 857-866 (2006).
4. H. Schellekens, N. Casadevall, Immunogenicity of recombinant human proteins: Causes and consequences, J. Neurol. Suppl. 251, 4-9 (2004).
5. A. S. De Groot, D. W. Scott, Immunogenicity of protein therapeutics, Trends Immunol. 28, 482-490 (2007).
6. G. M. Bartelds, C. A. Wijbrandts, M. T. Nurmohamed, S. Stapel, W. F. Lems, L. Aarden, B. A. C. Dijkmans, P. P. Tak, G. J. Wolbink, Clinical response to adalimumab: Relationship to anti-adalimumab antibodies and serum adalimumab concentrations in rheumatoid arthritis, Ann. Rheum. Dis. 66, 921-926 (2007).
7. G. J. Wolbink, L. A. Aarden, B. A. C. Dijkmans, Dealing with immunogenicity of biologicals: Assessment and clinical relevance, Curr. Opin. Rheumatol. 21, 211-215 (2009).
8. R. Zori, J. A. Thomas, N. Shur, W. B. Rizzo, C. Decker, O. Rosen, M. Li, B. Schweighardt, K. Larimore, N. Longo, Induction, titration, and maintenance dosing regimen in a phase 2 study of pegvaliase for control of blood phenylalanine in adults with phenylketonuria, Mol. Genet. Metab. 125, 217-227 (2018).
9. J. Thomas, H. Levy, S. Amato, J. Vockley, R. Zori, D. Dimmock, C. O. Harding, D. A. Bilder, H. H. Weng, J. Olbertz, M. Merilainen, J. Jiang, K. Larimore, S. Gupta, Z. Gu, H. Northrup, Pegvaliase for the treatment of phenylketonuria: Results of a long-term phase 3 clinical trial program (PRISM), Mol. Genet. Metab. 124, 27-38 (2018).

1. A composition comprising a polypeptide comprising an engineered Fms Related Receptor Tyrosine Kinase 3 Ligand (Flt3L) protein.

2. The composition of claim 1, wherein the polypeptide comprises an albumin protein.

3. The composition of claim 2, wherein the engineered Flt3L protein is connected to the albumin protein.

4. The composition of claim 3, wherein the polypeptide is a fusion of the engineered Flt3L protein and the albumin.

5. The composition of claim 3, wherein the engineered Flt3L protein is conjugated to the albumin protein.

6. The composition of any of claims 2-5, wherein the albumin protein is a Mouse Serum Albumin (MSA) protein.

7. The composition of claim 6, wherein the Flt3L-MSA fusion protein comprises an amino acid sequence that is at least 90% identical to SEQ ID NOs: 58 and 60.

8. The composition of any of claims 2-5, wherein the albumin protein is a Human Serum Albumin (HSA) protein.

9. The composition of claim 8, wherein the Flt3L-HSA fusion protein comprises an amino acid sequence that is at least 90% identical to SEQ ID NOs: 59 and 61.

10. The composition of claim 1, wherein the Flt3L protein is fused to a Fc domain of an IgG1 (Flt3L-Fc).

11. The composition of claim 10, wherein the Fc protein is a mouse Fc protein.

12. The composition of claim 11, wherein the Flt3L-Fc fusion protein comprises an amino acid sequence that is at least 90% identical to SEQ ID NOs: 58 and 62.

13. The composition of claim 10, wherein the Fc protein is a human Fc protein.

14. The composition of claim 13, wherein the Flt3L-Fc fusion protein comprises an amino acid sequence that is at least 90% identical to SEQ ID NOs: 59 and 63.

15. The composition of any one of claims 1-14, wherein the Flt3L protein is a mouse Flt3L protein.

16. The composition of any one of claims 1-14, wherein the Flt3L protein is a human Flt3L protein.

17. The composition of any one of claims 1-16, wherein the composition does not comprise rapamycin.

18. The composition of any one of claims 1-17, wherein the composition further comprises an immunogenic biomolecule and/or immunogenic cell therapy.

19. The composition of claim 18, wherein the immunogenic biomolecule is a nucleic acid, protein, or virus, or a combination thereof.

20. The composition of claim 19, wherein the nucleic acid is DNA, RNA, or a combination thereof.

21. The composition of claim 19, wherein the nucleic acid is an siRNA, miRNA, gRNA, mRNA, lincRNA, cDNA, gene or gene fragment, expression construct, or plasmid, or a combination thereof.

22. The composition of claim 19, wherein the virus is adenovirus, adeno-associated virus, lentivirus, or retrovirus, or a combination thereof.

23. The composition of claim 19, wherein the protein is an enzyme, an antigen binding protein, an antibody or antibody fragment, a cytokine, a chemokine, a ligand, a receptor, or binding protein, or a combination thereof.

24. The composition of claim 19, wherein the cell therapy comprises T-cells, B-cells, dendritic cells, NK or iNK cells, other hematopoietic cells, epithelial cells, neuronal or nerve cells, stem cells, pluripotent cells, cardiac cells, skeletal cells, smooth muscle cells, skin cells, endothelial cells, fat cells, pancreatic cells, or bone cells, or a combination thereof.

25. The composition of any one of claims 1-24, wherein the composition comprises or consists essentially of one or more of the Flt3L protein, Flt3L fusion protein, an immunogenic biomolecule, and an immunogenic cell therapy.

26. The composition of any one of claims 1-25, wherein the composition consists of the Flt3L protein and/or Flt3L fusion protein.

27. A method of treatment comprising: administering to a subject in need thereof an effective amount of an engineered Flt3L protein of any one of claims 1-26.

28. A method for inducing immunotolerance in a subject in need thereof comprising, the method comprising administering to the subject an engineered Flt3L protein of any one of claims 1-26.

29. The method of claim 27 or 28, wherein the FLT3L polypeptide comprises a fusion protein comprising: a FLT3L polypeptide linked to a serum protein.

30. The method of claim 29, wherein the serum protein comprises albumin.

31. The method of claim 30, wherein the albumin comprises human serum albumin or mouse serum albumin.

32. The method of any one of claims 29-31, wherein the serum protein comprises the amino acid sequence of SEQ ID NO:60 or 61, or an amino acid sequence having at least 80% sequence identity to SEQ ID NO:60 or 61.

33. The method of any one of claims 27-32, wherein the FLT3L polypeptide comprises the amino acid sequence of one of SEQ ID NOs: 40-50, 58 or 59 or an amino acid sequence having at least 80% sequence identity to one of SEQ ID NOs: 40-50, 58 or 59.

34. The method of any one of claims 27-33, wherein the FLT3L polypeptide comprises a fusion protein comprising: a FLT3L extracellular domain operably linked to an immunoglobulin fragment crystallizable region (Fc region).

35. The method of claim 34, wherein at least 5 amino acids are truncated from the C-terminus of the FLT3L extracellular domain; and/or the Fc region does not comprise a hinge region.

36. The method of claim 34 or 35, wherein the FLT3L extracellular domain is a human FLT3L extracellular domain or derived from a human FLT3L extracellular domain.

37. The method of any one of claims 34-36, wherein the fusion protein is capable of binding to human FLT3.

38. The method of any one of claims 34-37, wherein the FLT3L extracellular domain is from FLT3L isoform 1.

39. The method of any one of claims 34-37, wherein the FLT3L extracellular domain is from FLT3L isoform 2.

40. The method of any one of claims 34-39, wherein the FLT3L extracellular domain does not comprise the amino acid sequence PTAPQ.

41. The method of any one of claims 34-40, wherein at least 6, 7, 8, 9, 10, 11, 12, 13, 14 or 15 amino acids are truncated from the C-terminus of the FLT3L extracellular domain.

42. The method of any one of claims 34-41, wherein the FLT3L extracellular domain does not comprise the amino acid sequence APTAPQ (SEQ ID NO:29), TAPTAPQ (SEQ ID NO: 30), ATAPTAPQ (SEQ ID NO:31), EATAPTAPQ (SEQ ID NO:32), or LEATAPTAPQ (SEQ ID NO:33).

43. The method of any one of claims 34-42, wherein the FLT3L extracellular domain does not comprise the amino acid sequence PTAPQPP (SEQ ID NO:34), APTAPQPP (SEQ ID NO: 35), TAPTAPQPP (SEQ ID NO:36), ATAPTAPQPP (SEQ ID NO:37), EATAPTAPQPP (SEQ ID NO:38), or LEATAPTAPQPP (SEQ ID NO:39).

44. The method of any one of claims 34-43, wherein the FLT3L extracellular domain comprises an N-terminal signal peptide.

45. The method of any one of claims 34-44, wherein the FLT3L extracellular domain comprises amino acid substitutions at one or more of the following amino acid positions: H8Y, K84E, N100, S102, N123 and S125, wherein the amino acid residue positions are with reference to SEQ ID NOs: 1-18, 21-27 or 40-50.

46. The method of any one of claims 34-45, wherein the FLT3L extracellular domain comprises one or more of the following amino acid substitutions: H8Y, K84E, S102A, and/or S125A; wherein the amino acid residue positions are with reference to SEQ ID NOs: 1-18, 21-27 or 40-50.

47. The method of any one of claims 34-46, wherein one or both of serine residues at positions 102 and 125 are substituted to alanine, wherein the amino acid residue positions are with reference to SEQ ID NOs: 1-18, 21-27 or 40-50.

48. The method of any one of claims 34-47, wherein the Fc region is from a human IgG1, IgG2, IgG3 or IgG4.

49. The method of any one of claims 34-48, wherein the Fc region comprises a human IgG1 isotype and comprises one or more amino acid substitutions in the Fc region at a residue position selected from the group consisting of: N297A, N297G, N297Q, N297G, D265A, L234A, L235A, C226S, C229S, P238S, E233P, L234V, P238A, A327Q, A327G, P329A, P329G, K322A, L234F, L235E, P331S, T394D, A330L, M252Y, S254T, T256E, M428L, N434S, T366W, T366S, L368A, Y407V, and any combination thereof, wherein the numbering of the residues is according to EU numbering.

50. The method of claim 49, wherein the Fc region comprises a human IgG1 isotype and comprises one or more amino acid substitutions in the Fc region at a residue position selected from the group consisting of: L234A, L234V, L234F, L235A, L235E, P331S, and any combination thereof, wherein the numbering of the residues is according to EU numbering.

51. The method of any one of claims 34-48, wherein the Fc region comprises a human IgG4 isotype and comprises one or more amino acid substitutions in the Fc region at a residue position selected from the group consisting of: E233P, F234V, F234A, L235A, G237A, E318A, S228P, L235E, T394D, M252Y, S254T, T256E, N297A, N297G, N297Q, T366W, T366S, L368A, Y407V, M428L, N434S, and any combination thereof, wherein the numbering of the residues is according to EU numbering.

52. The method of claim 51, wherein the Fc region comprises a human IgG4 isotype and comprises one or more amino acid substitutions in the Fc region at a residue position selected from the group consisting of: F234V, F234A, L235A, L235E, S228P, and any combination thereof, wherein the numbering of the residues is according to EU numbering.

53. The method of any one of claims 34-52, wherein the Fc region comprises the following amino acids at the indicated positions (EU index numbering): Tyrosine at position 252, threonine at position 254 and glutamic acid at position 256 (YTE); or Leucine at position 428 and serine at position 434 (LS).

54. The method of any one of claims 34-53, wherein the FLT3L extracellular domain comprises the amino acid sequence of one of SEQ ID NOs: 40-50, or an amino acid sequence having at least 80% sequence identity to one of SEQ ID NOs: 40-50.

55. The method of any one of claims 34-54, wherein the Fc region comprises the amino acid sequence of one of SEQ ID NOs: 51-55, 62, and 63, or an amino acid sequence having at least 80% sequence identity to one of SEQ ID NOs: 51-55, 62, and 63.

56. The method of any one of claims 34-55, wherein the fusion protein comprises the amino acid sequence of one of SEQ ID NOs: 1-27, or an amino acid sequence having at least 80% sequence identity to one of SEQ ID NOs: 1-27.

57. The method of any one of claims 34-56, wherein the Fc region is from a human IgG1 and does not comprise a hinge region.

58. The method of claim 57, wherein the C-terminus of the FLT3L extracellular domain is not truncated.

59. The method of any one of claims 34-58, wherein the Fc region is derived from a human IgG1 isotype and does not comprise a hinge region, e.g., does not the amino acid sequence EPKSCDKTHTCPPCP (SEQ ID NO:56) or EPKSCDKTHTCPPCPAPELL (SEQ ID NO:57).

60. The method of any one of claims 34-58, wherein the Fc region is from a human IgG4 and at least 5 amino acids are truncated from the C-terminus of the FLT3L extracellular domain.

61. The method of any one of claims 34-60, wherein the Fc region comprises a hinge region.

62. The method of any one of claims 34-61, wherein the Fc region is derived from a human IgG4 isotype and wherein at least 5 amino acids are truncated from the C-terminus of the FLT3L extracellular domain, e.g., wherein the FLT3L extracellular domain does not comprise the amino acid sequence PTAPQ.

63. The method of any one of claims 27-62, wherein the subject in need is also administered an exogenous antigen wherein the exogenous antigen comprises a therapeutic biomolecule and/or cell therapy.

64. The method of any one of claims 27-62, wherein the subject is one that has been or is being treated with an exogenous antigen wherein the exogenous antigen comprises a therapeutic biomolecule and/or cell therapy.

65. The method of claim 63 or 64, wherein the therapeutic biomolecule and/or cell therapy comprises an immunogenic biomolecule and/or immunogeneic cell therapy.

66. The method of any one of claims 63-65, wherein the therapeutic biomolecule is a nucleic acid, protein, or virus, or a combination thereof.

67. The method of claim 66, wherein the nucleic acid is DNA, RNA, or a combination thereof.

68. The method of claim 66, wherein the nucleic acid is an siRNA, miRNA, gRNA, mRNA, lincRNA, cDNA, gene or gene fragment, expression construct, or plasmid, or a combination thereof.

69. The method of claim 66, wherein the virus is adenovirus, adeno-associated virus, lentivirus, or retrovirus, or a combination thereof.

70. The method of claim 66, wherein the protein is an enzyme, an antigen binding protein, an antibody or antibody fragment, a cytokine, a chemokine, a ligand, a receptor, or binding protein, or a combination thereof.

71. The method of claim 63, wherein the cell therapy comprises T-cells, B-cells, dendritic cells, NK or iNK cells, other hematopoietic cells, epithelial cells, neuronal or nerve cells, stem cells, pluripotent cells, cardiac cells, skeletal cells, smooth muscle cells, skin cells, endothelial cells, fat cells, pancreatic cells, or bone cells, or a combination thereof.

72. The method of any one of claims 63-71, wherein both the exogenous antigen and engineered Flt3L protein are provided to the subject within a 72 hour period.

73. The method of any one of claims 63-72, wherein the treatment prevents and/or inhibits induction of an immune response (e.g., promotes tolerogenesis) in the subject against the exogenous antigen.

74. The method of claim 63-73, wherein the subject has an increased and/or enhanced tolerance to the exogenous antigen without reduction, inhibition, and/or blunting of other productive immune responses.

75. The method of any one of claims 63-74, wherein excessive inflammation (e.g., life threatening and/or capable of creating permanent physiological damage) in a subject in response to the exogenous antigen is avoided.

76. The method of any one of claims 63-75, wherein the exogenous antigen and/or engineered Flt3L protein is provided orally, nasally, mucosally, intravenously, and/or subcutaneously.

77. The method of any one of claims 27-76, wherein the providing of the Flt3L protein prevents and/or treats an autoimmune condition in the subject.

78. The method of any one of claims 27-77, wherein the subject is a mammal.

79. The method of any one of claims 27-78, wherein the subject is a human.

80. The method of any of claims 63-79, wherein the engineered Flt3L protein is provided at the same time or within one day of the exogenous antigen.
